(19) United States
(12) Patent Application Publication
(10) Pub. No.: US 2024/0268874 A1
(43) Pub. Date: Aug. 15, 2024

(54) SYSTEMS AND METHODS FOR PROVIDING A SALVATION PLATE

(71) Applicants: Brock A. Liden, Pickerington, OH (US); Spencer T. Brown, Leesburg, IN (US); Parker R. Lawson, Hebron, OH (US)

(72) Inventors: Brock A. Liden, Pickerington, OH (US); Spencer T. Brown, Leesburg, IN (US); Parker R. Lawson, Hebron, OH (US)

(21) Appl. No.: 18/439,458

(22) Filed: Feb. 12, 2024

Related U.S. Application Data
(60) Provisional application No. 63/525,126, filed on Jul. 5, 2023.
(60) Provisional application No. 63/444,935, filed on Feb. 11, 2023.
(60) Provisional application No. 63/445,239, filed on Feb. 13, 2023.

(51) Int. Cl. A61B 17/80; A61B 17/56

(52) U.S. Cl. CPC A61B 17/8085; A61B 17/8033; A61B 17/8052; A61B 17/8061; A61B 17/809; A61B 2017/564

(57) ABSTRACT

In some cases, the salvation plate includes an elongated plate having: a first end, a second end that is disposed opposite to the first end. In some cases, the plate defines a first fastener opening that is disposed at the first end and that is configured to allow a first fastener to extend therethrough so as to enter into a bone covered by the plate at a first angle that falls within a first maximum range of angular motion. In some cases, the plate also defines a second fastener opening that is configured to allow a second fastener to extend therethrough to enter into the bone at a second angle that falls within a second maximum range of angular motion that is smaller than the first maximum range of angular motion. Additional implementations are described herein.

### CROSS-REFERENCE TO RELATED APPLICATIONS

This application claims priority to each of: U.S. Provisional Application Ser. No. 63/444,935 (Attorney Docket No. 35023.3), which is entitled SYSTEMS AND METHODS FOR PROVIDING A SALVATION PLATE, and which was filed on Feb. 11, 2023; U.S. Provisional Application Ser. No. 63/445,239 (Attorney Docket No. 35023.4), which is entitled SYSTEMS AND METHODS FOR PROVIDING A SALVATION PLATE, and which was filed on Feb. 13, 2023; and U.S. Provisional Application Ser. No. 63/525,126 (Attorney Docket No. 35023.6), which is entitled SYSTEMS AND METHODS FOR PROVIDING A SALVATION PLATE, and which was filed on Jul. 5, 2023; the entire disclosures of which are incorporated herein by reference.

### BACKGROUND

### Field

The described systems and methods relate to the treatment of bones and joints. In particular, the described systems and methods relate to a salvation plate that can be placed over one or more deteriorating, broken, diseased, malformed, collapsing, or otherwise damaged bones or joints and that can be tightened to the bones or joints with one or more screws, pins, staples, nails, bolts, threaded engagements, anchors, wires, or other fasteners. In this manner, the plate can provide compression to various pieces of the bones and joints to help them heal, fuse, mend, strengthen, and for any other suitable purpose. Additionally, in some cases, one or more screws or other fasteners that extend through the plate are configured to extend through the plate in a wide range of angles. Thus, in some cases, the described salvation plate allows a screw or other fastener to be driven through the plate at an angle that allows the fastener to grab pieces of bone that could otherwise not be grabbed were the fastener not able to be inserted at that specific angle.

### Background and Related Art

Many times, when a bone breaks, collapses, or fractures, it is damaged in such a way that it is difficult for the bone to heal, at least without surgery where one or more screws or rods are coupled to the bone to hold it together. Similarly, in some cases in which one or more joints are severely damaged, the bones on each side of the joint are held together with one or more screws or rods to allow the joint to heal.

While the use of screws and rods to fix bones and joints is relatively common, such methods are not necessarily without their shortcomings. Indeed, in some cases, bones and joints can be so damaged that it can be difficult, if not impossible, to treat such bones and joints with screws and rods alone.

Thus, while techniques currently exist that are used to help heal damaged bones and joints, challenges still exist, including those listed above. Accordingly, it would be an improvement in the art to augment or even replace current techniques with other techniques.

### SUMMARY

The described systems and methods relate to the treatment of bones and joints. In particular, the described systems and methods relate to a salvation plate that can be placed over one or more deteriorating, broken, diseased, malformed, collapsing, or otherwise damaged bones or joints and that can be tightened to the bones or joints with one or more screws, pins, staples, nails, bolts, threaded engagements, anchors, wires, or other fasteners. In this manner, the plate can provide compression to various pieces of the bones and joints to help them heal, fuse, mend, strengthen, and for any other suitable purpose. Additionally, in some cases, one or more screws or other fasteners that extend through the plate are configured to extend through the plate in a wide range of angles. Thus, in some cases, the salvation plate allows a screw or other fastener to be driven through the plate at an angle that allows the fastener to grab pieces of bone that could otherwise not be grabbed were the fastener not able to be inserted at that specific angle.

The salvation plate (or plate) can include any suitable component or characteristic that allows it to function as described herein. For instance, the plate can have any suitable shape that allows the plate to function as intended. Indeed, in some implementations, the plate comprises one or more portions that are substantially flat, planar, curved, rounded, convex, concave, branched, lobed, polygonal, narrowed, broadened, necked, symmetrical, asymmetrical, chamfered, raised, depressed, bent, angled, formed (e.g., manually or in any other suitable manner) to meet the needs of one or more bone interfaces, or that have any other suitable shape. In some cases, for instance, a portion of the salvation plate comprises a planar section. In some cases, however, a portion of the salvation plate comprises a convex external surface and a concave internal surface that is disposed opposite to the convex surface such that the concave internal surface is configured to wrap or otherwise extend at least partially around one or more bones that are covered by the plate.

In some cases, the plate defines one or more first fastener openings (e.g., or anchor opening) that are each configured to allow a first fastener (or anchor) to extend through the plate so as to enter into one or more bones that are covered by the plate at a first angle that falls within a first maximum range of angular motion. In some such cases, the first range of angular motion is relatively large (e.g., between 0 degrees and 210 degrees (or within any subrange thereof) with respect to a lower portion of the plate (e.g., internal surface of the plate that is disposed distally, or in any other suitable location, with respect to the first fastener opening). As the first fastener can extend through the first fastener opening at a wide variety of angles, the first fastener can be oriented into many different angles to allow the first fastener to grab (and get “purchase” in) portions of the bones that would be difficult (if not impossible) to grab if the angular range of motion of the first fastener in the first fastener opening were more limited.

Where the plate defines one or more first fastener openings, the plate can define any suitable number of openings, including, 1, 2, 3, 4, 5, 6, 7, 8, or more. Additionally, such openings can be disposed in any suitable locations that allow the plate to function as described herein. In this regard, some examples of suitable locations for the first fastener opening include a proximal, distal, medial, lateral, central, corner, posterior, anterior, inferior, superior, or any other suitable portion (or portions) of the plate. Indeed, in some cases, one or more first fastener openings are disposed at or adjacent to a center of the plate. Moreover, in some cases, one or more first fastener openings (or anchor fastener openings or anchor openings) are disposed at a proximal end portion of the plate. In some such cases, by driving a first fastener (an anchor, or any suitable fastener that extends through the first fastener opening or anchor opening) through the first (or anchor) faster opening with the first fastener (or an anchor or an anchor fastener) extending at least somewhat in a proximal direction into the bone, the first fastener can pull one or more bones, bone parts, or joints together in a proximal direction.

In some cases, the plate also defines one or more second fastener openings (or civility holes) that are each configured to allow a second fastener (or a civility fastener, or any suitable fastener that extends through the second (or civility) fastener opening) to extend through the plate and to enter into the bone at a second angle that falls within a second range of angular motion, which is smaller than the first range of angular motion. While second fasteners can be inserted through the second fastener openings at any suitable angle, in some implementations, the plate is configured (e.g., by its shape; by the shape, size, configuration, placement, or any other suitable characteristic of the second fastener openings; or in any other suitable manner) to direct two or more of the second fasteners into bone at converging angles to securely lock the plate to the bone.

Where the plate defines one or more second fastener openings, such openings can be disposed in any suitable location or locations, including at a distal, proximal, medial, lateral, corner, central, posterior, anterior, inferior, superior, or any other suitable portion of the plate. Indeed, in some cases, most (if not all) of the second fastener openings are disposed closer to a distal end portion of the plate than to a proximal end portion of the plate than is one or more of the first fastener openings.

These and other features and advantages of the described systems and methods will be set forth or will become more fully apparent in the description that follows and in the appended claims. The features and advantages may be realized and obtained by means of the instruments and combinations particularly pointed out in the appended claims. Furthermore, the features and advantages of the described systems and methods may be learned by the practice thereof or will be obvious from the description. as set forth hereinafter.

### BRIEF DESCRIPTION OF THE DRAWINGS

In order that the manner in which the above recited and other features and advantages of the described systems and methods are obtained, a more particular description of the described systems and methods will be rendered by reference to specific embodiments thereof, which are illustrated in the appended drawings. Understanding that the drawings (including any measurements, wording, specifications, images, quantities, and other information provided therein) depict only representative embodiments of the described systems and methods and are not, therefore, to be considered as being limiting in scope. In this light, the described systems and methods will be set forth and explained with additional specificity and detail through the use of the accompanying drawings in which:

FIGS. 1A-1B each illustrate an elevation, cross-sectional view of a salvation plate that comprises fastener openings that provide fasteners with various degrees of ingress angles, in accordance with some representative embodiments;

FIGS. 2-4 each illustrate an elevation cross-sectional view of the salvation plate, in accordance with some representative embodiments;

FIGS. 5-9 each illustrate a plan view of a different representative embodiment of the salvation plate;

FIG. 10 illustrates an elevation view of an end of the salvation plate with two fasteners, in accordance with some embodiments;

FIG. 11-12B each illustrate an elevation, cross-sectional view of the salvation plate, in accordance with some embodiments;

FIGS. 13A-14E illustrate different views of the salvation plate, in accordance with a representative embodiment;

FIGS. 14F-14G illustrate various views of the salvation plate being disposed adjacent to bone, in accordance with some embodiments;

FIGS. 14H-24B illustrate various view of the salvation plate, in accordance with some embodiments;

FIGS. 25A-25X illustrate various views of fasteners that can be used with the salvation plate, in accordance with some embodiments;

FIGS. 26A-30 illustrate various catches that are configured to retain fasteners that are seated in the salvation plate, in accordance with some embodiments;

FIG. 31A illustrates a flowchart depicting a method for using the salvation plate, in accordance with a representative embodiment;

FIGS. 31B-31L illustrate various views of a guide and jig for countersinking bone to help seat the salvation plate, in accordance with some embodiments;

FIG. 31M illustrates a plan view of a pin guide, in accordance with some embodiments;

FIG. 31N illustrates a side view of the pin guide, wherein the pin guide is placed on a bone and a pin is driven through the pin guide and into the bone, in accordance with some embodiments;

FIG. 31O illustrates a side view of the pin that has been driven into the bone with a countersinking object disposed on the pin and extending into the bone (after the countersinking object has removed some bone), in accordance with some embodiments;

FIGS. 31P and 31R each illustrate a side view of a fastener that comprises threads and a bone removal tool, in accordance with some embodiments; and

FIG. 31Q illustrates a perspective, partial-cutaway view depicting the salvation plate being coupled to a bone, with a fastener (e.g., an anchor fastener) extending into a solidifying material in the bone.

### DETAILED DESCRIPTION

The described systems and methods relate to the treatment of bones and joints. In particular, the described systems and methods relate to a salvation plate that can be placed over one or more deteriorating, broken, diseased, malformed, collapsing, or otherwise damaged bones or joints and that can be tightened to the bones or joints with one or more screws, pins, staples, nails, bolts, threaded engagements, anchors, wires, or other fasteners. In this manner, the plate can provide compression to various pieces of the bones and joints to help them heal, fuse, mend, strengthen, and for any other suitable purpose. Additionally, in some cases, one or more screws or other fasteners that extend through the plate are configured to be able to extend through the plate in a wide range of angles. Thus, in some cases, the salvation plate allows a screw or other fastener to be driven through the plate at an angle that allows the fastener to grab pieces of bone that could otherwise not be grabbed were the fastener not able to be inserted at that specific angle.

In many cases, as medical personnel perform surgeries, it can be difficult to find suitable portions of bone that can be coupled together (e.g., where the bone is fractured or otherwise damaged) with a just a screw or rod. This can be especially true in serious bone fractures and in joints that are severely damaged. In this regard, some embodiments of the described salvation plate allow one or more screws or other fasteners to be moved to a wide variety of angles to allow the fasteners to: grab (or get “purchase” in) bone that would otherwise be difficult to capture, and force the salvation plate to pull or otherwise move one or more bones or joints to help provide compression and strength to the bones or joint (e.g., to help them to fuse together, heal, or for any other suitable purpose). Thus, in some cases in which a bone or joint could not otherwise be fused, the described plate allows medical personnel to save, salvage, or otherwise provide salvation to the bone or joint.

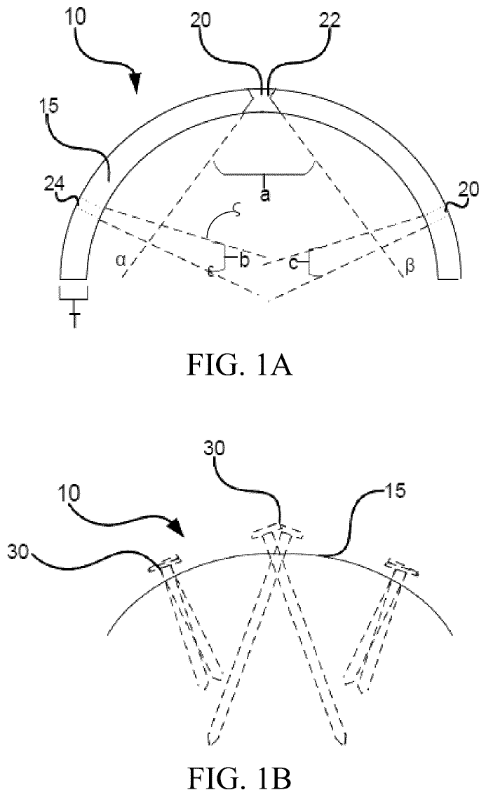

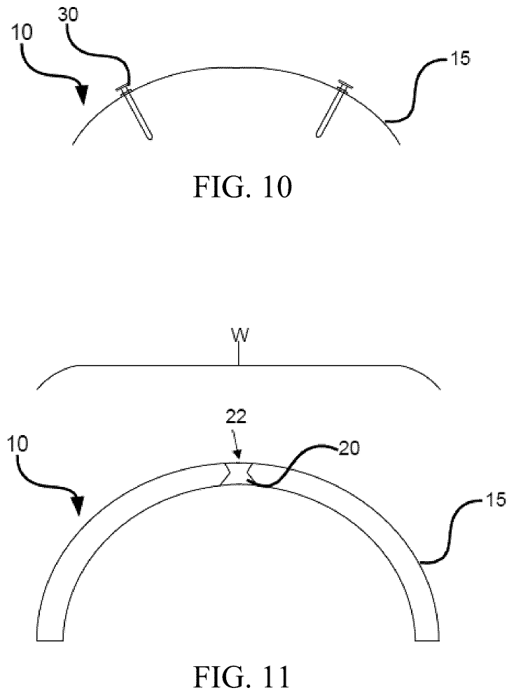

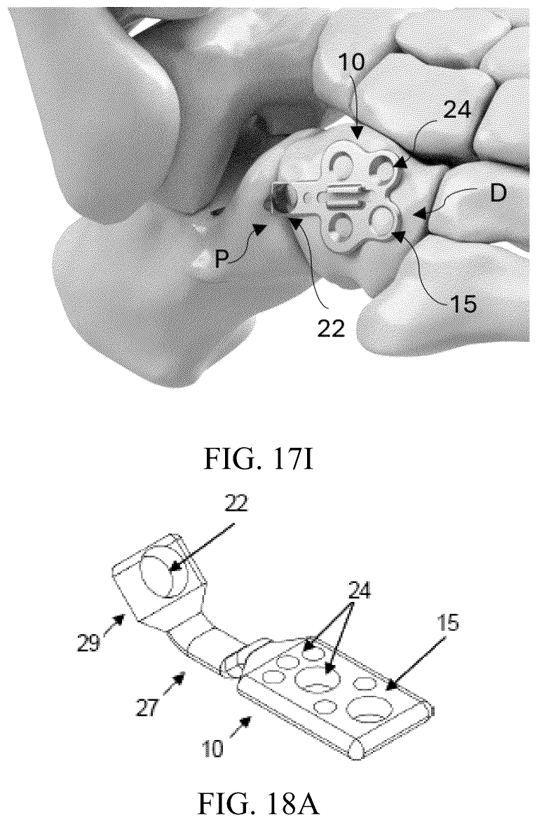

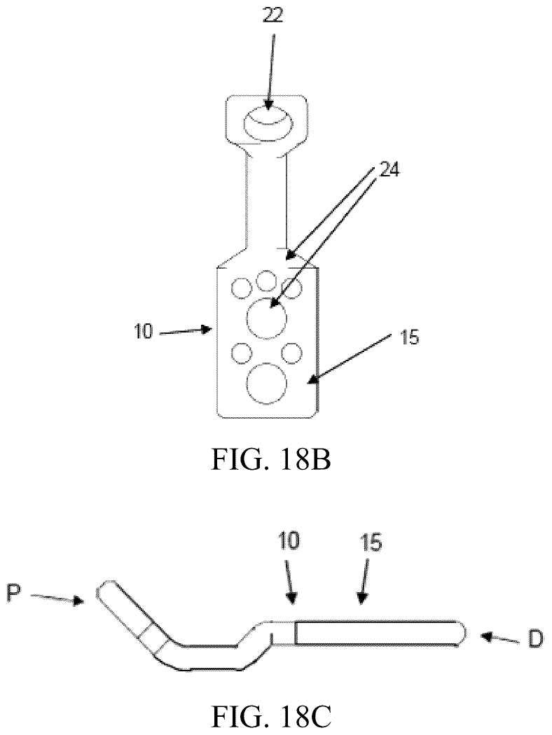

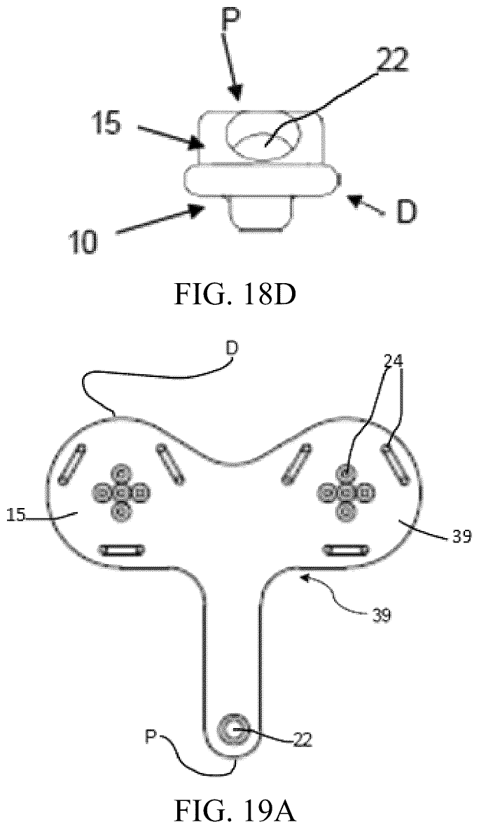

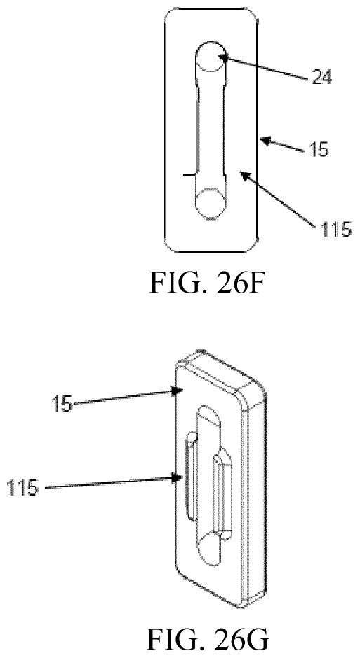

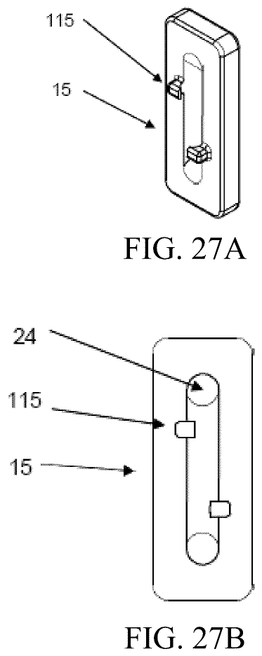

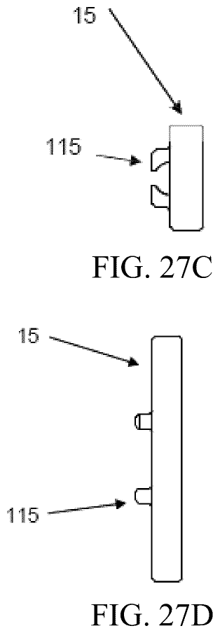

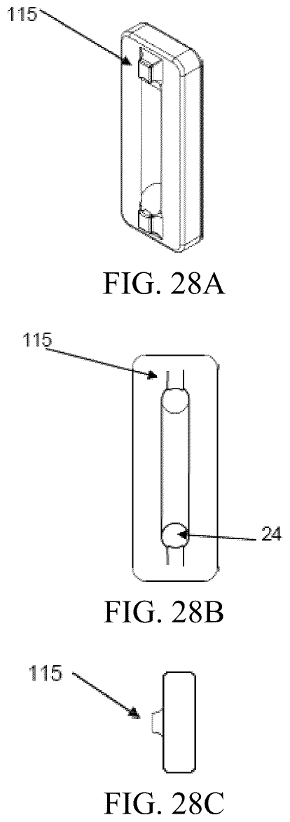

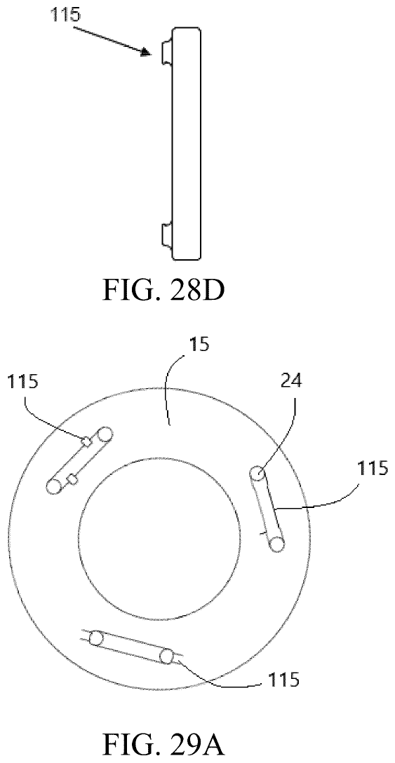

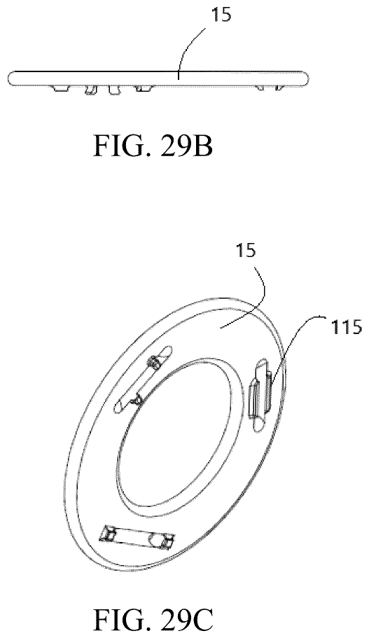

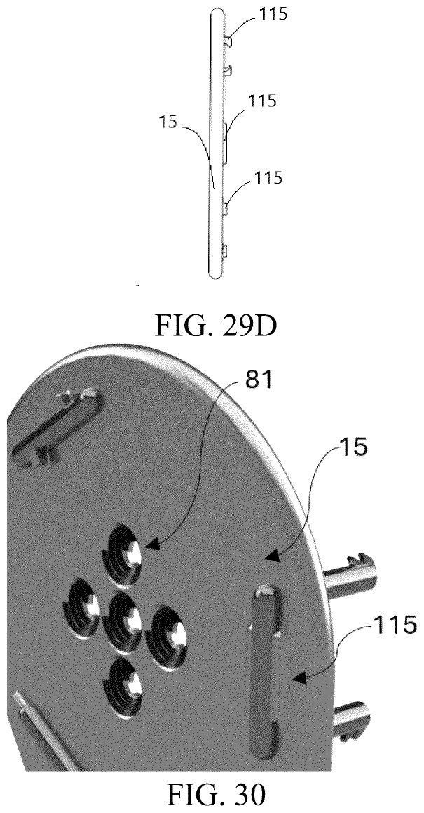

While the described salvation plate can comprise any suitable component or characteristic that allows it to function as intended, FIGS. 1-30 illustrate some embodiments in which the salvation plate system 10 comprises one or more salvation plates 15; fastener openings 20 (e.g., first fastener openings 22, second fastener openings 24, or any other suitable type of fastener openings); fastener spacers 25; recessed portions 27; curved, bent, or angled portions 29; fasteners 30; projections 35; or any other suitable component that allows the salvation plate to be set over one or more bones or joints and to be fastened to the bones to apply compression or strength to the bones or joints to help the bones or joints to heal, fuse, mend, be stronger, move, or for any another suitable purpose.

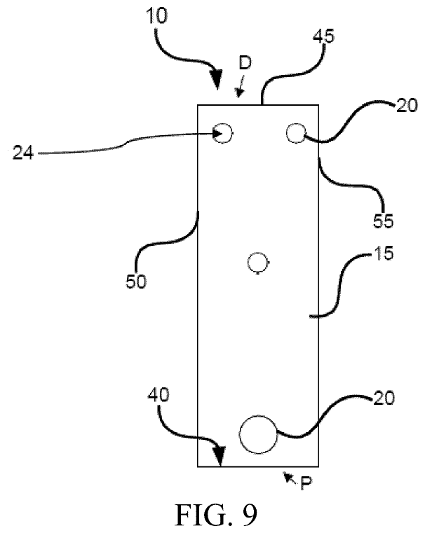

With respect to the salvation plate 15, the plate can comprise any suitable characteristic that allows it to function as described herein. Indeed, in accordance with some embodiments, FIG. 9 shows that the salvation plate 15 comprises a first end 40, a second end 45, a first lateral side 50, and a second lateral side 55. In this regard, while the first and second ends of the plate can be configured to be placed in any suitable orientation in a patient, in accordance with some embodiments, the first end is configured to be disposed proximally (or closer to a trunk of the body) and the second end is configured to be disposed more distally in the patient. Hence, several of the drawings use P (for proximal) to identify a potential proximal end of the plate and D (for distal) to identify a potential distal end of the plate.

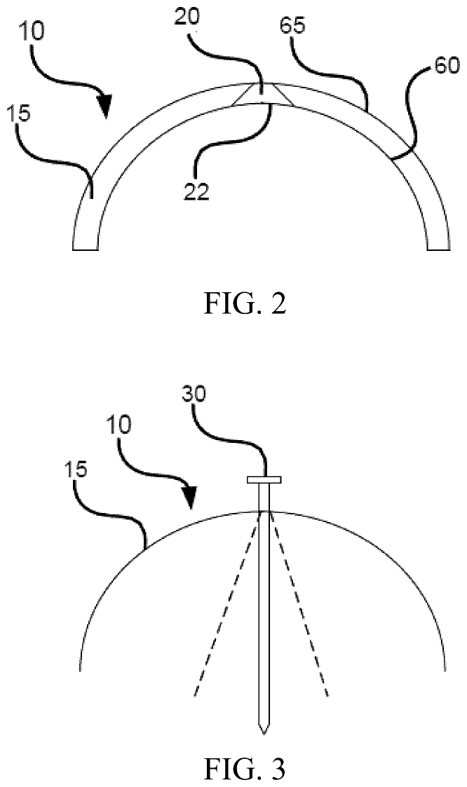

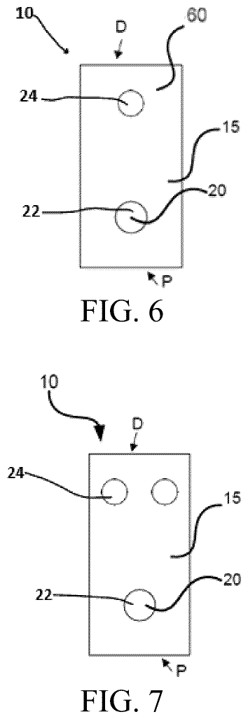

As another example of a suitable characteristic of the salvation plate 15, FIGS. 2 and 6 show that, in some embodiments, the plate 15 comprises a first (or internal) face 60 that is configured to be placed against one or more bones, and a second (or external) face 65 (not shown in FIG. 6) that is disposed substantially opposite to the first face 60.

The salvation plate 15 can have any suitable shape that allows it to be placed on and coupled to one or more bones or joints to help provide compression or to move to the bones or joints. Indeed, in some embodiments, when viewed from a plan view, the salvation plate is or comprises a portion that is circular, elliptical, triangular, square, rectangular, quadrilateral, hexagonal, pentagonal, polygonal, paddle-shaped, butterfly-shaped, lobed, branched, necked, hourglass shaped, symmetrical, asymmetrical, regular, irregular, configured to substantially match a shape of a bone, rounded, curved, elongated, shaped to have one or more narrowed sections, shaped to have one or more broadened sections, or that has any other suitable shape.

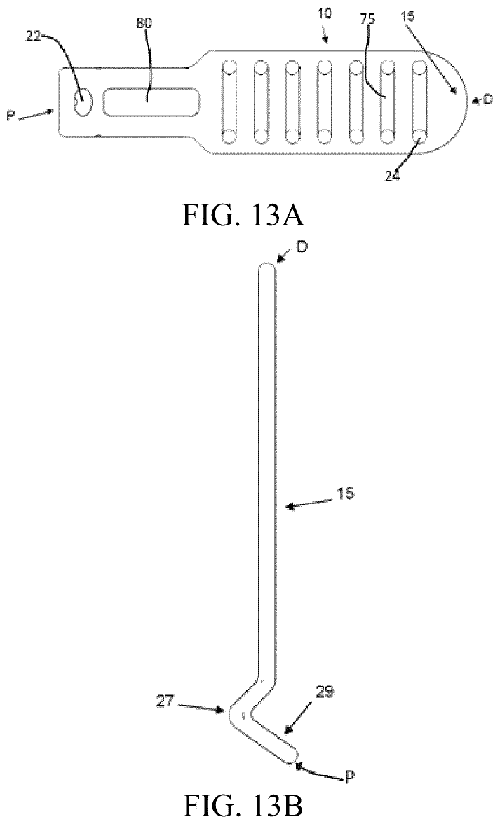

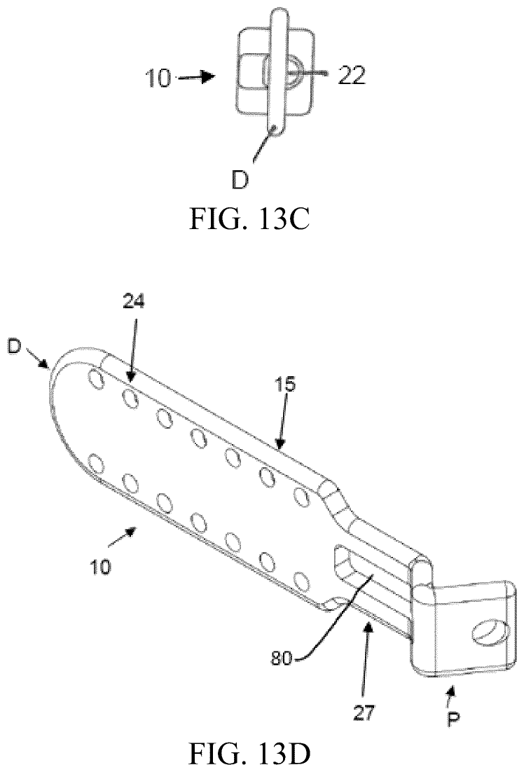

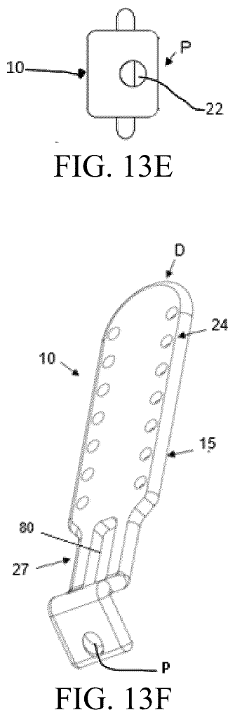

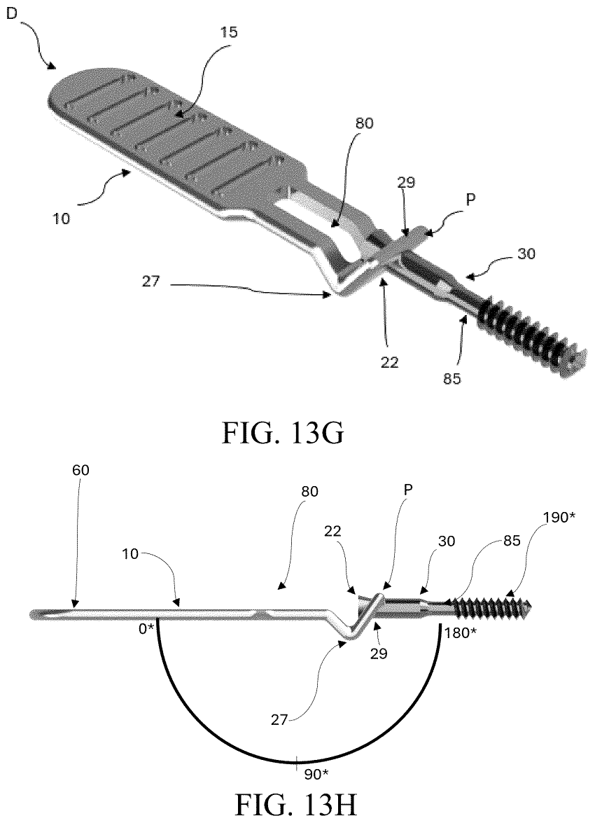

By way of non-limiting illustration, FIGS. 5-9 show that, in some embodiments, the salvation plate 15 is substantially rectangular. In this regard, while FIGS. 5-9 show some embodiments in which the salvation plate has relatively square corners, in some other embodiments, the corners are rounded, curved, chamfered, tapered, or comprise any other suitable shape. In some additional non-limiting illustrations, FIGS. 13A-14D, 14G-14O, and 18A-18C (among others) show some embodiments in which the plate 15 has a paddle-like shape (when seen from a plan view).

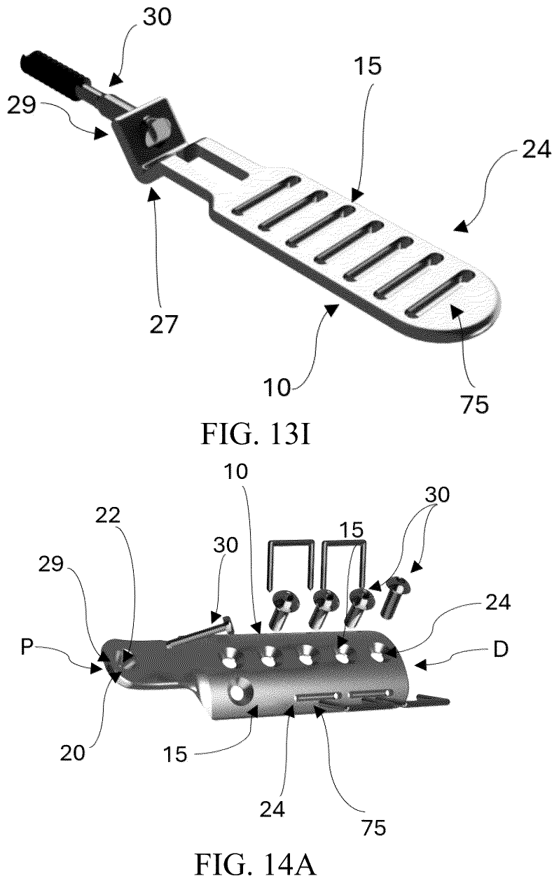

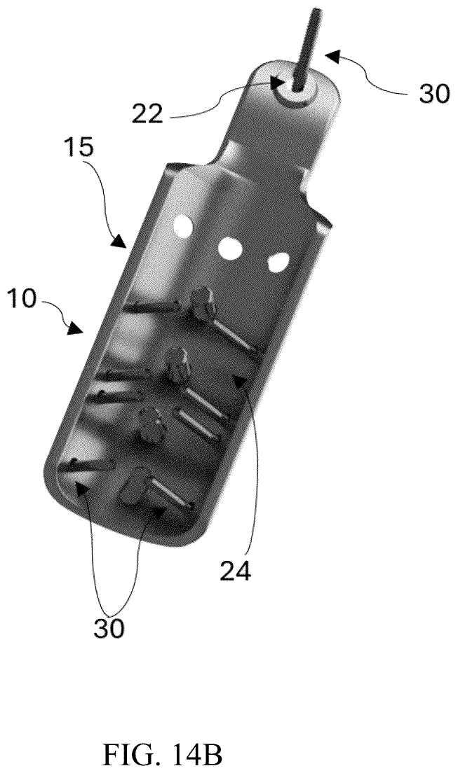

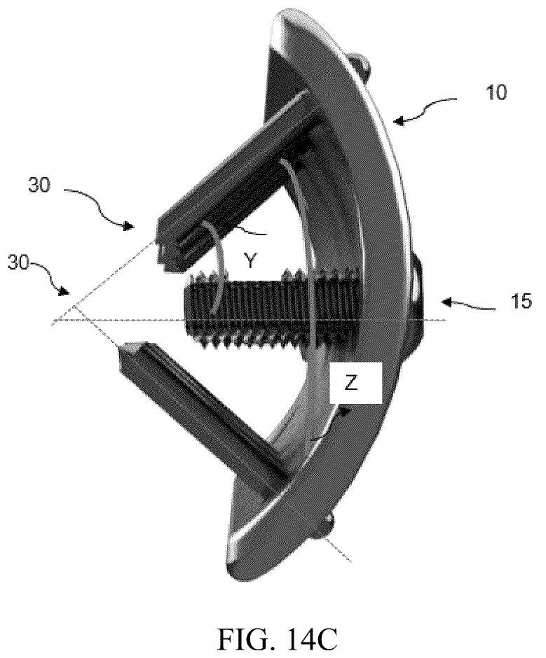

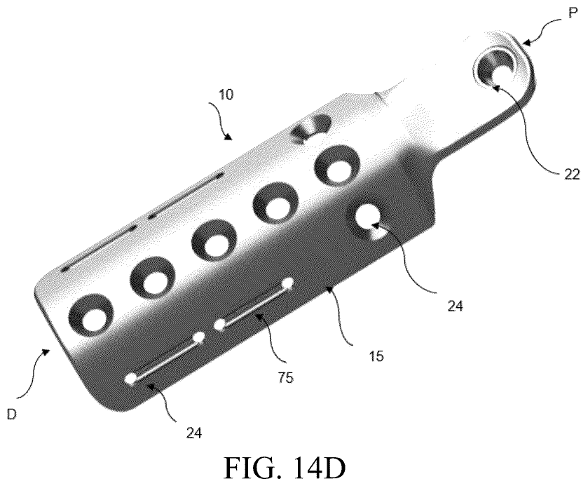

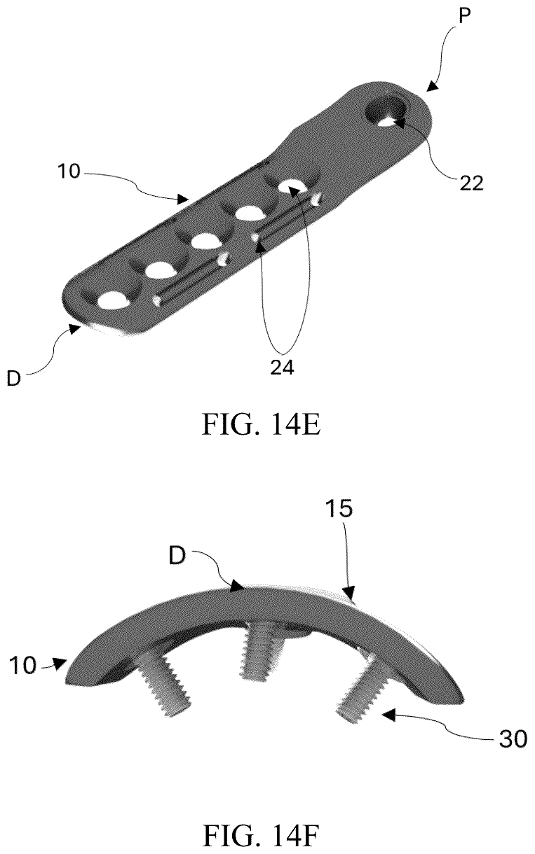

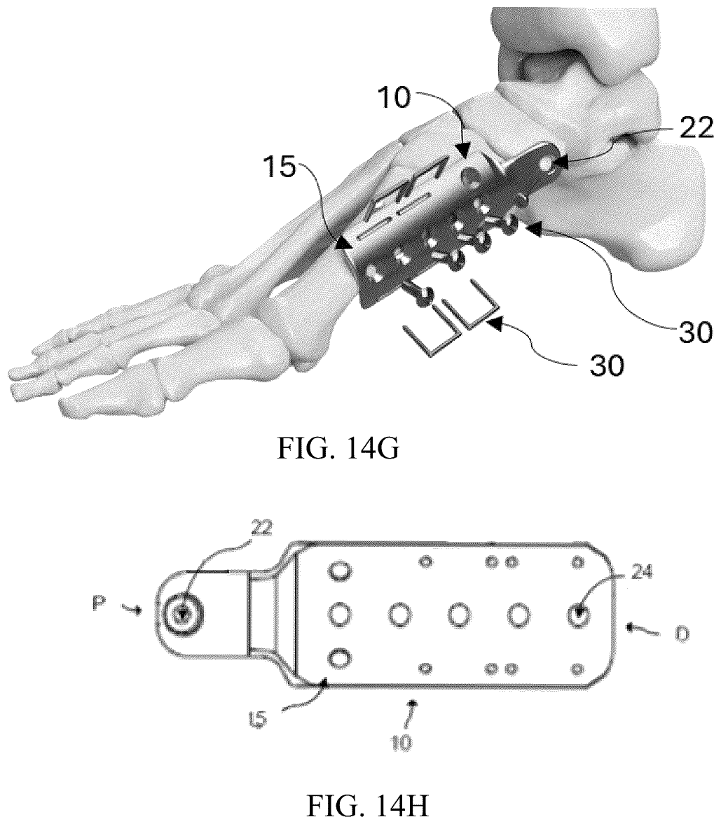

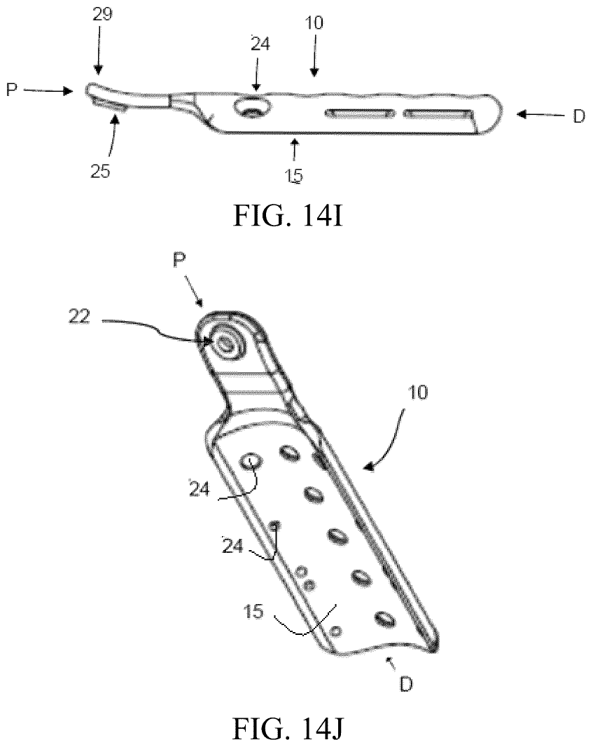

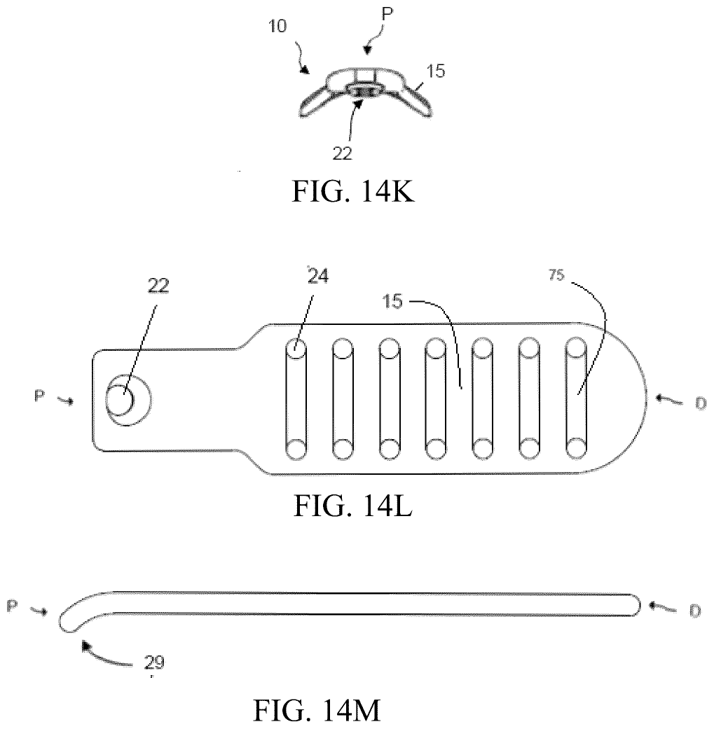

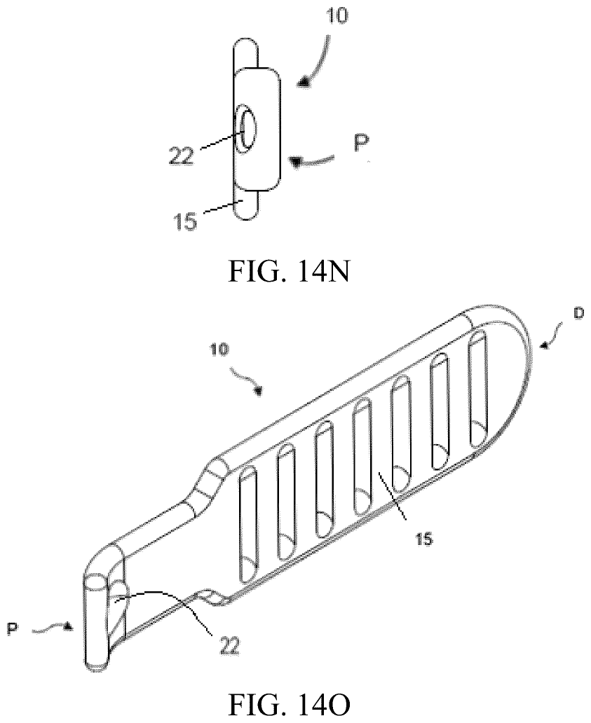

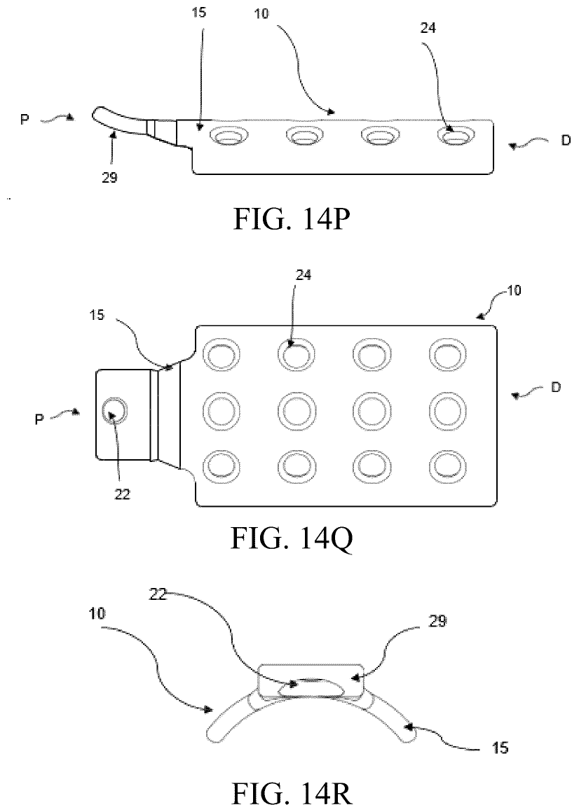

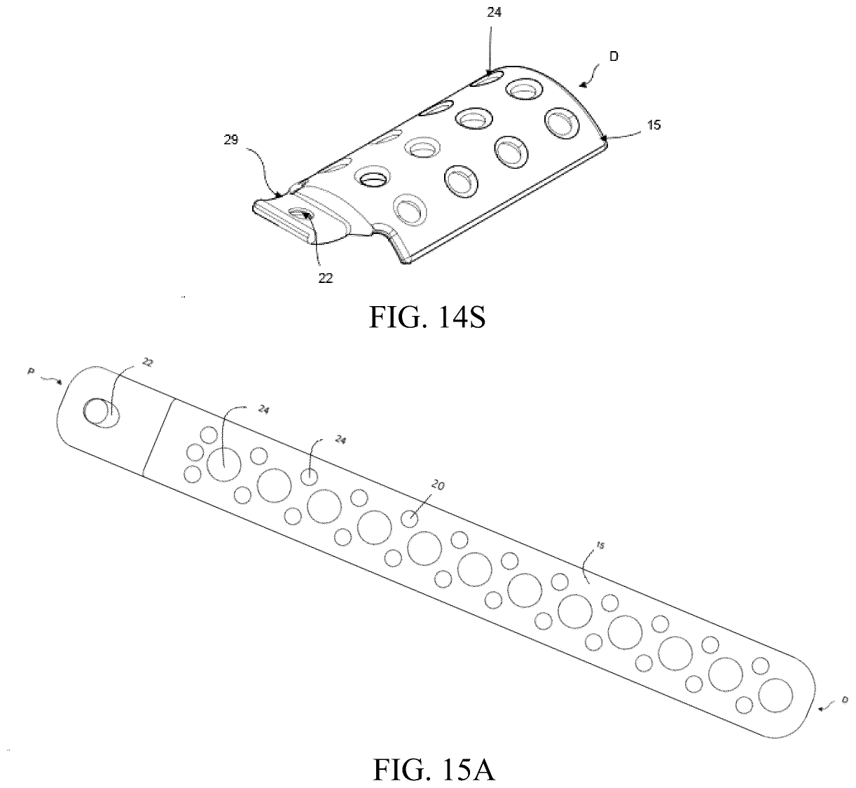

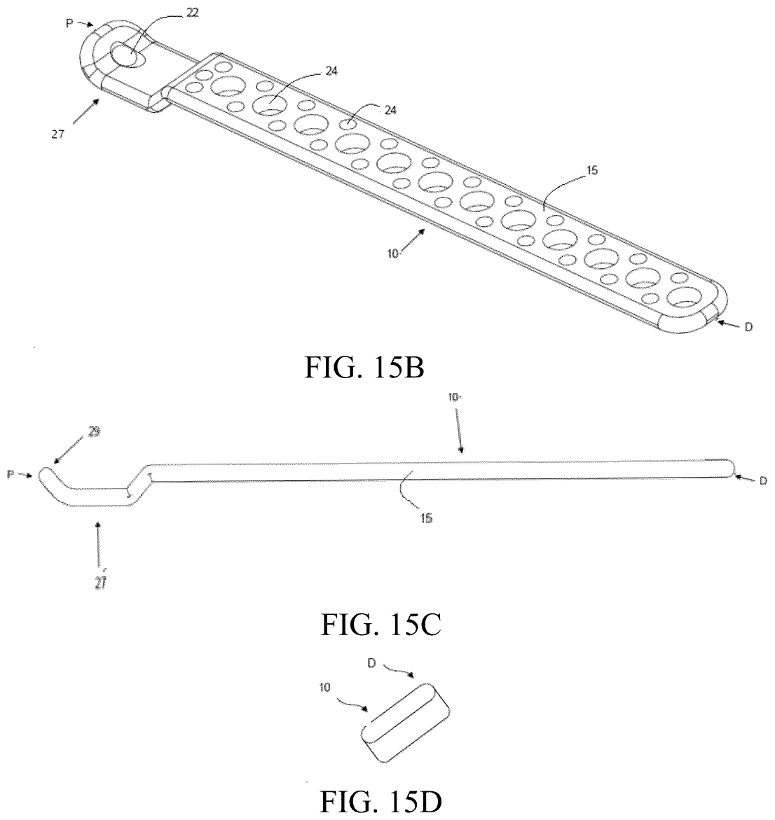

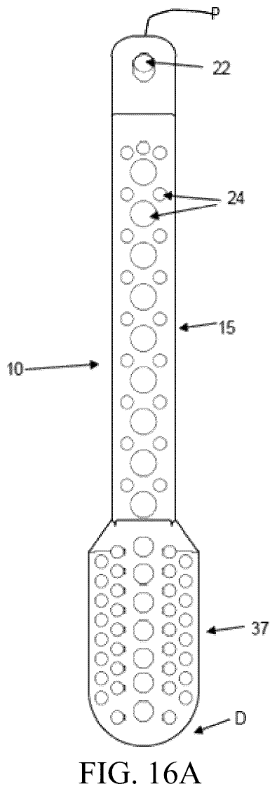

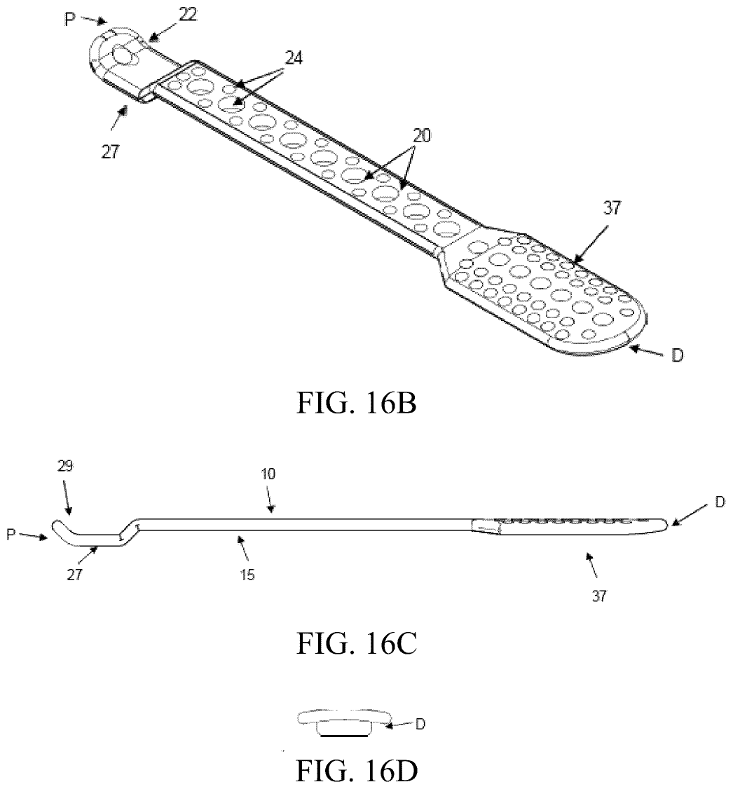

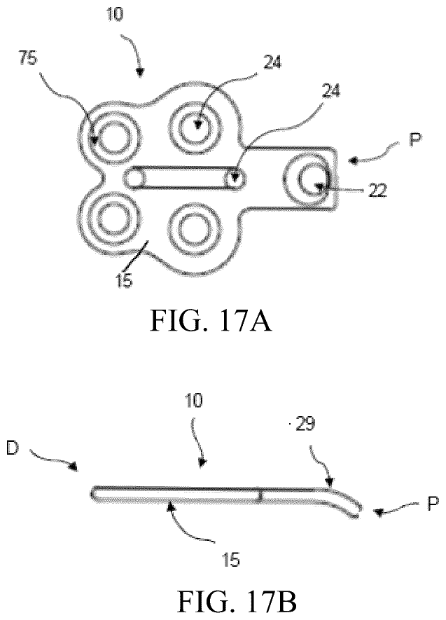

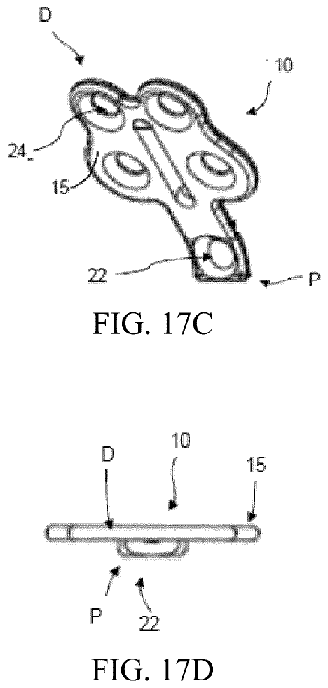

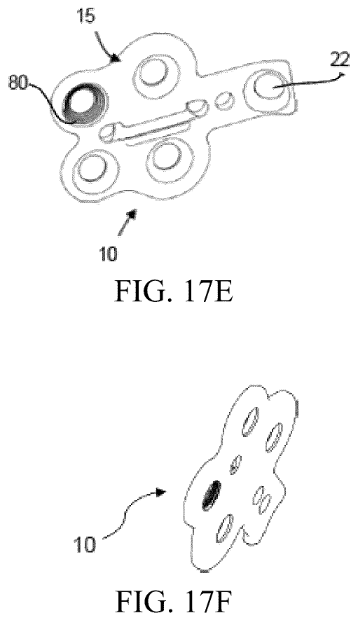

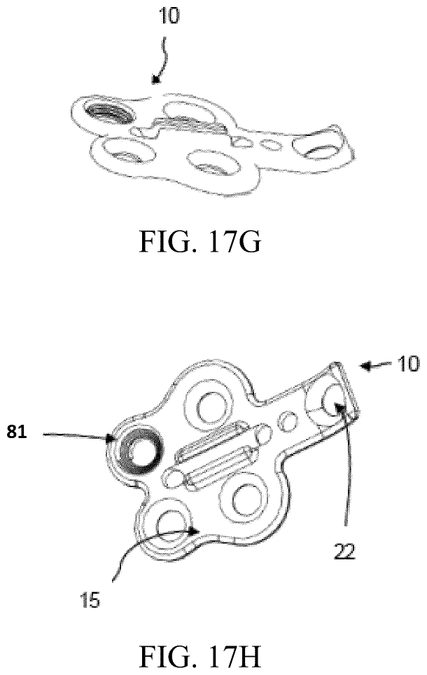

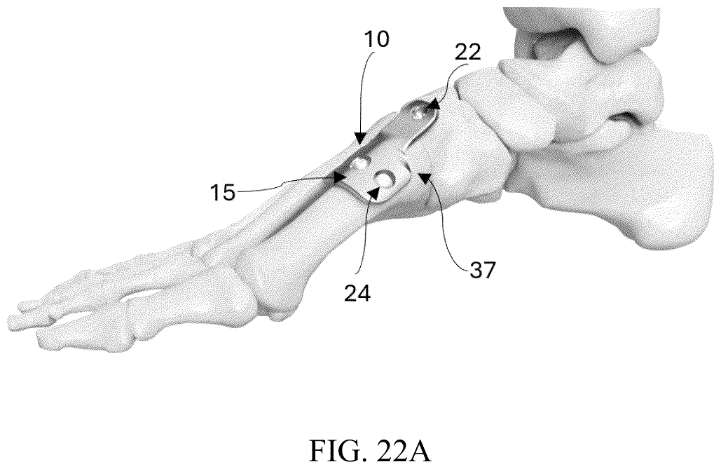

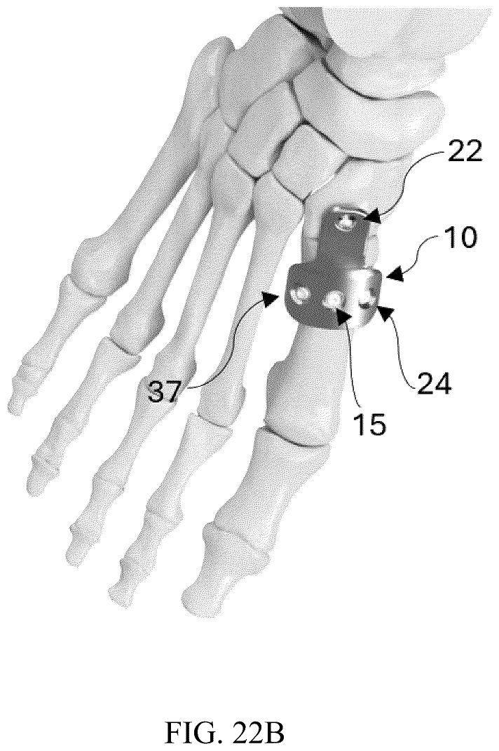

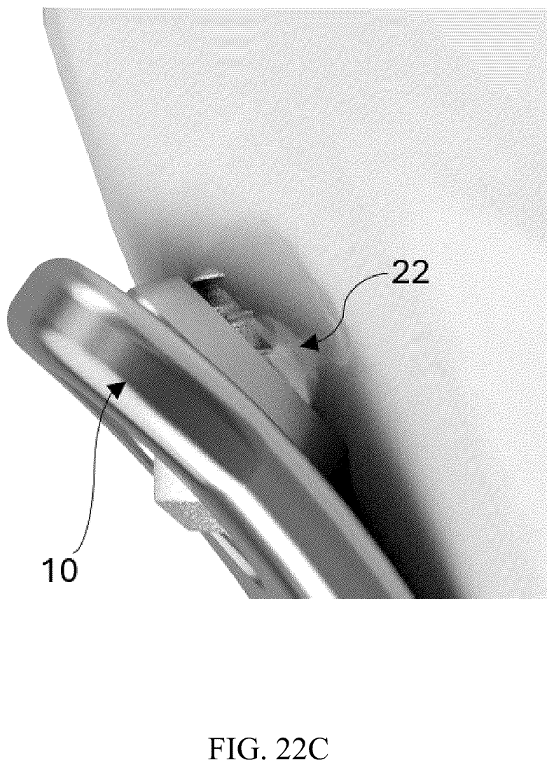

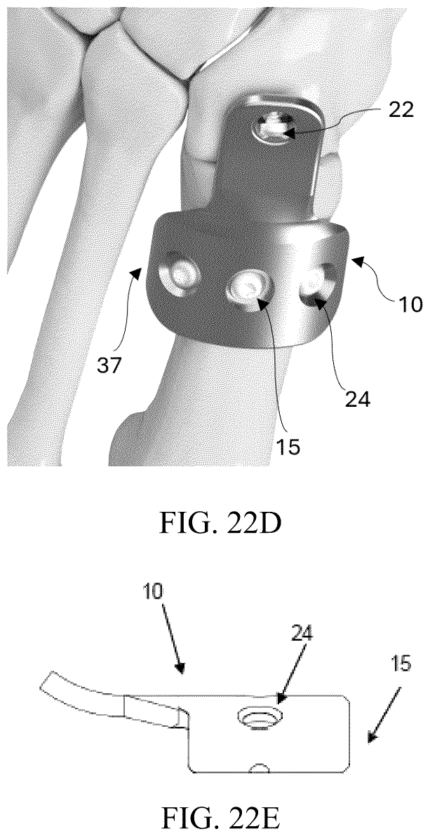

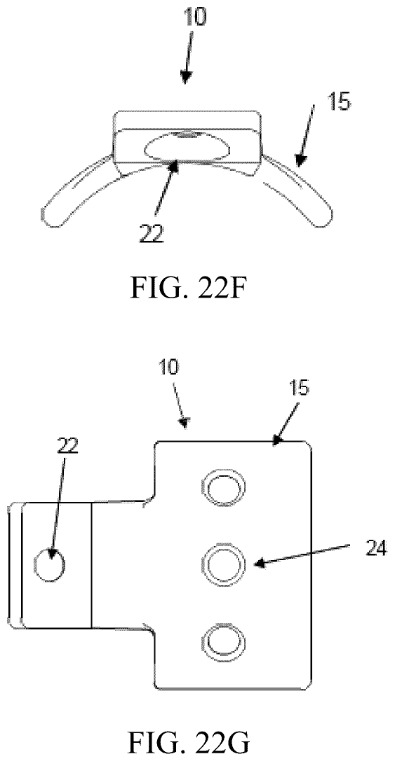

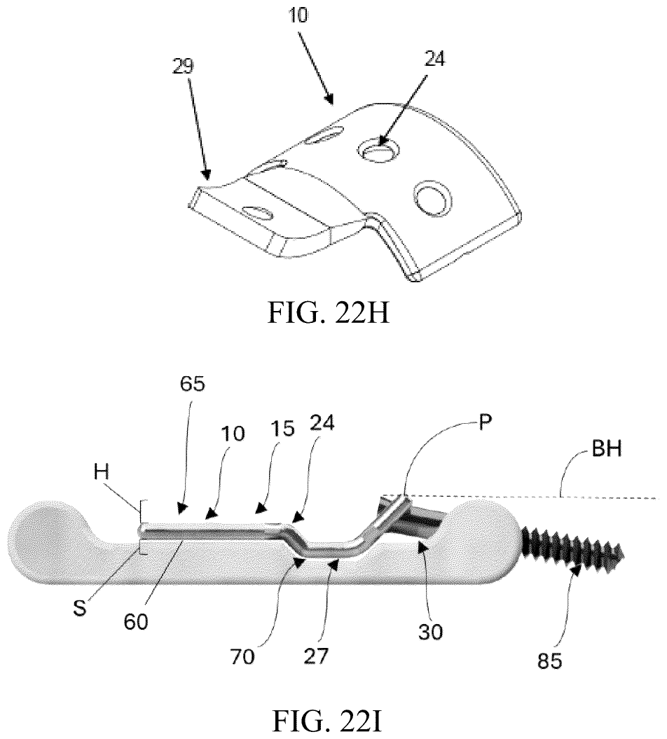

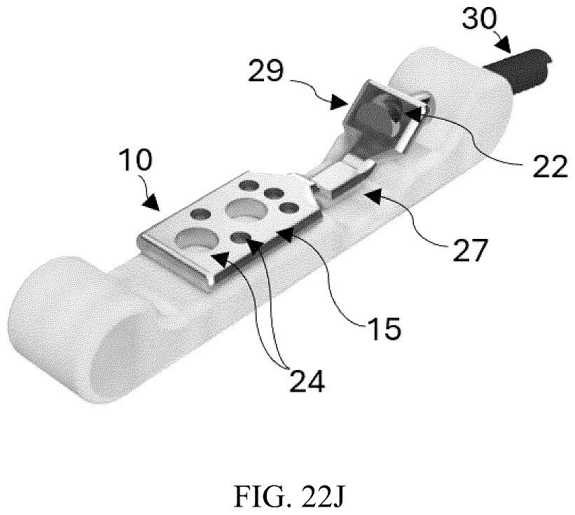

Moreover, FIGS. 14E and 15A-15B (among others) show some embodiments in which the plate 15 is shaped as an elongated strip or elongated plate (from a plan view). Additionally, FIGS. 16A-16D and 22A-22B show some embodiments in which the plate 15 has one or more broadened portions 37. In this regard, while broadened portions can be disposed in any suitable location and extend symmetrically or asymmetrically from any suitable portions of the plate, FIGS. 16A-16D and 22A-22B show some embodiments in which the broadened portion 37 is disposed at (or adjacent to) a distal end D of the plate 15 (e.g., for use on a fibula, metacarpal, metatarsal, or on any other suitable bone or bones). As yet another example of a suitable shape, FIGS. 17A-17E show some embodiments in which the plate 15 has bilateral symmetry (e.g., having somewhat of a butterfly-like shape).

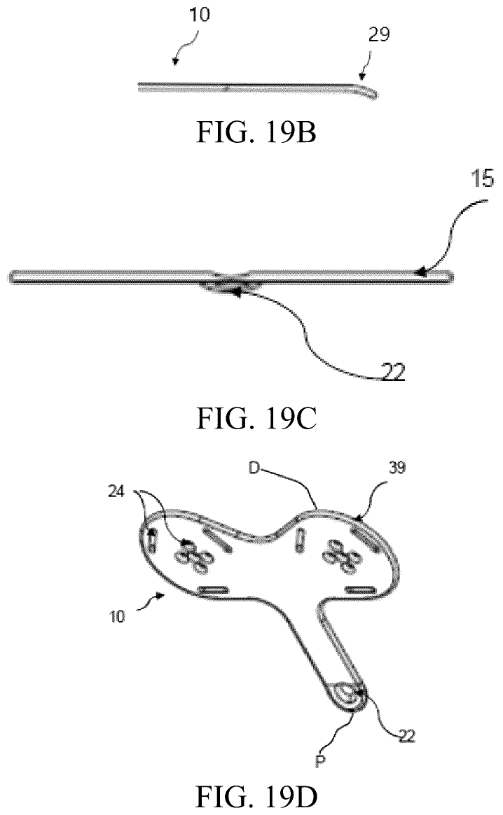

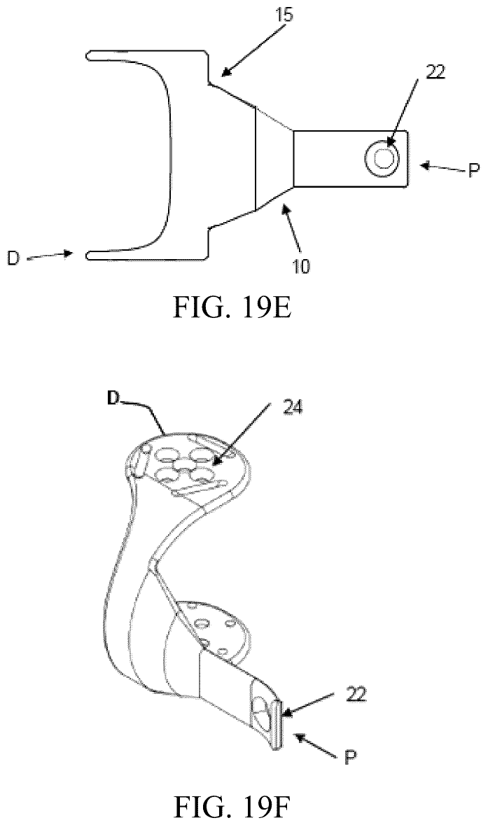

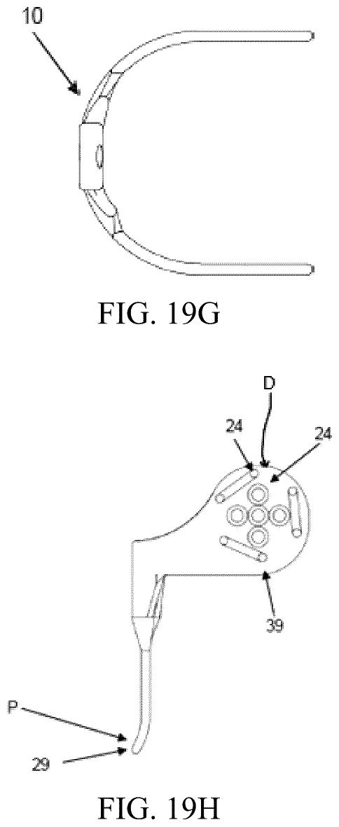

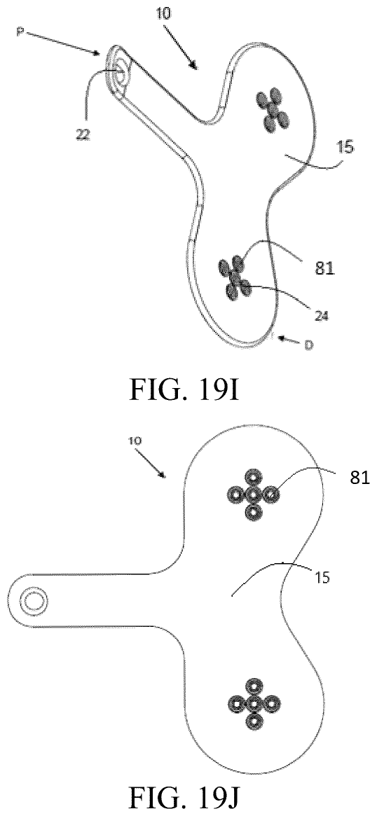

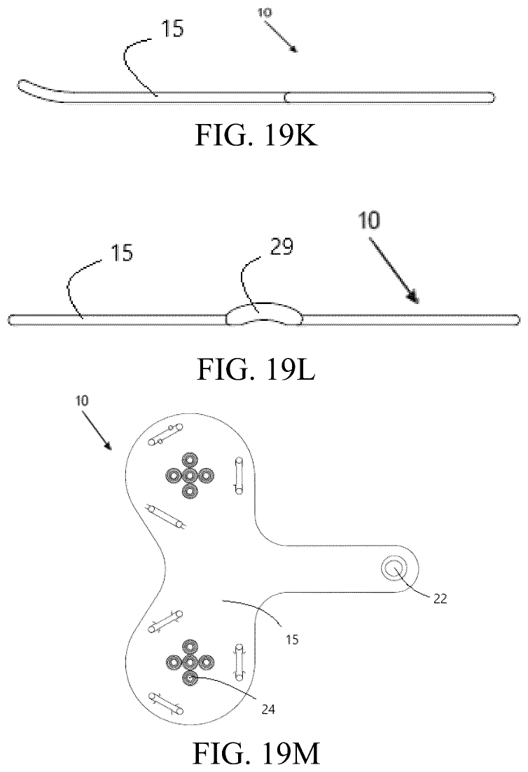

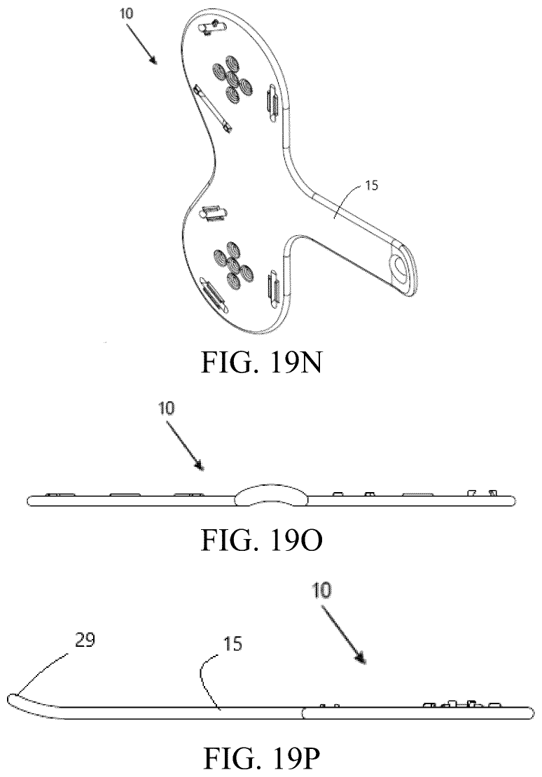

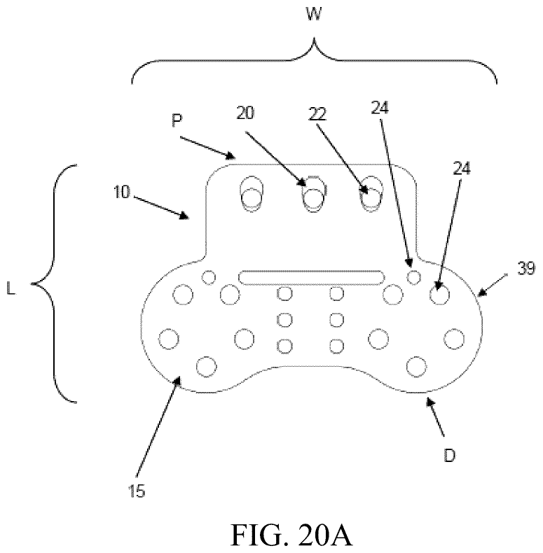

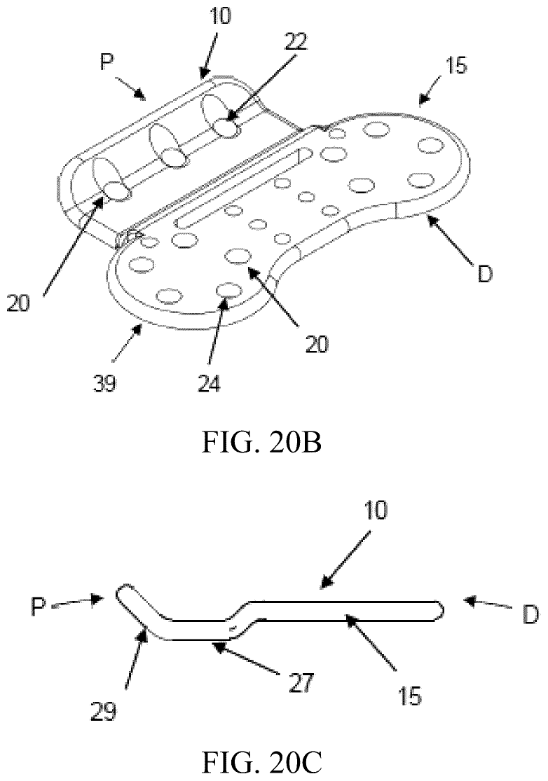

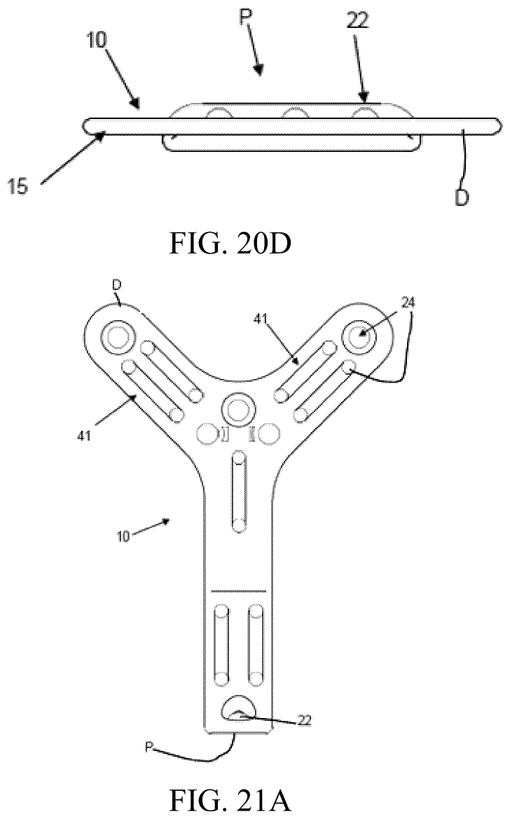

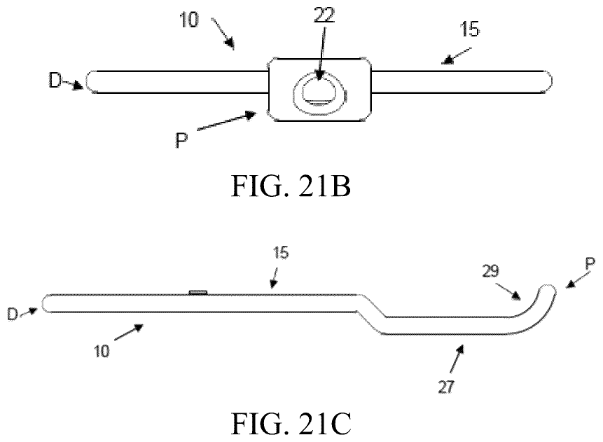

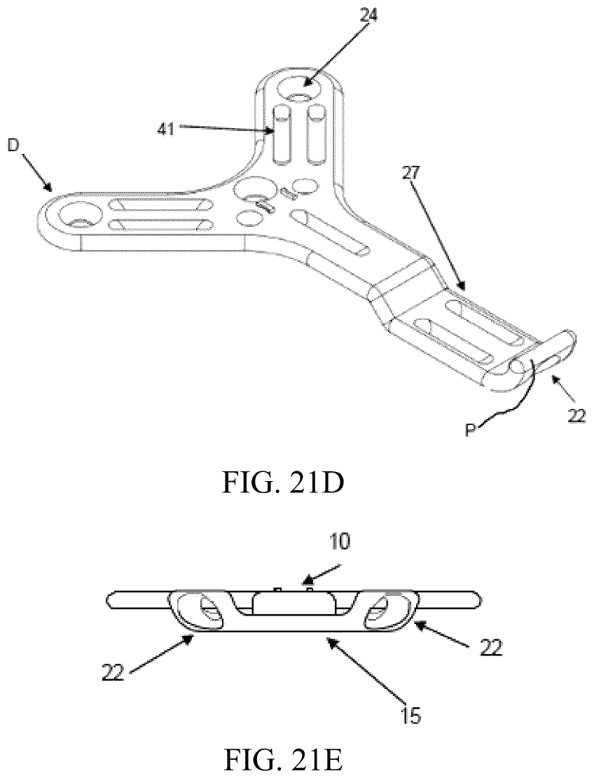

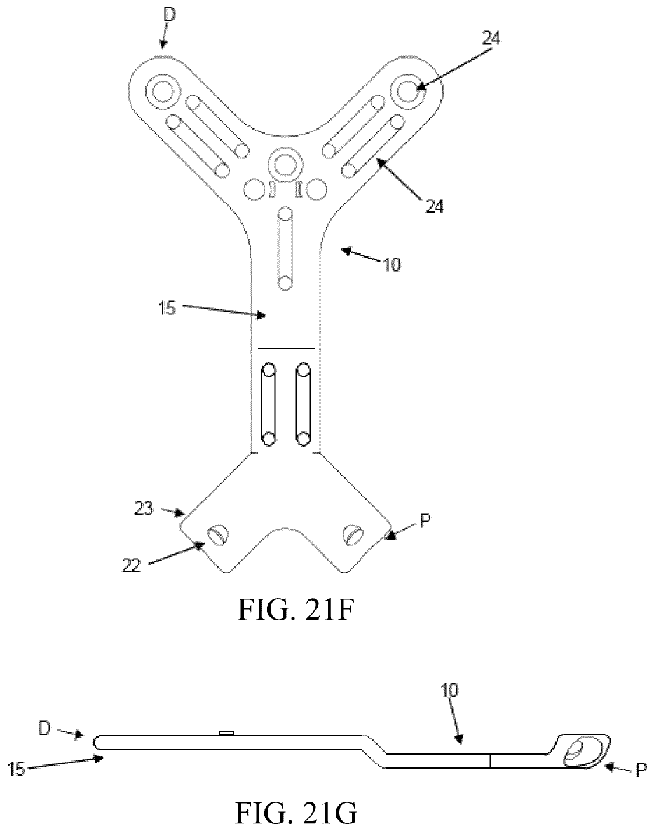

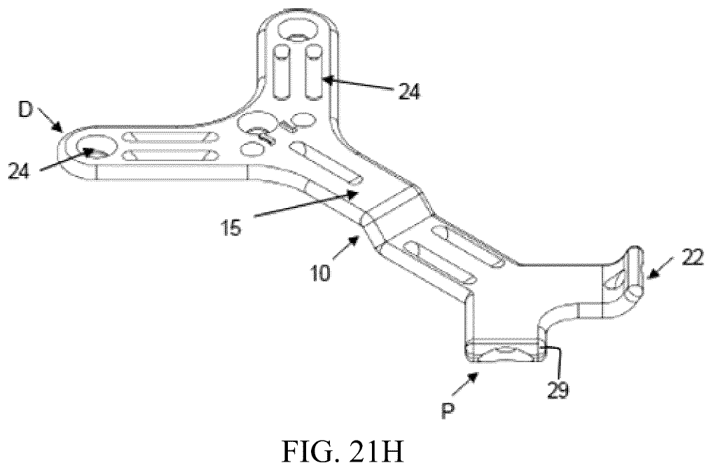

In another example, FIGS. 19A-20D (among others) show some embodiments in which the plate 15 has a lobed or hourglass configuration with one or more roundish, flattish lobes 39. Moreover, FIGS. 21A-21H show some embodiments in which the plate 15 has a branched configuration. In this regard, while the plate 15 can have any suitable number of branches that are disposed in any location on the plate 15, FIGS. 21A-21D show some embodiments in which the plate 15 comprises two branches 41 that are disposed at a distal end D of the plate 15. Similarly, FIG. 21G shows an embodiment in which a proximal end P of the plate is bifurcated.

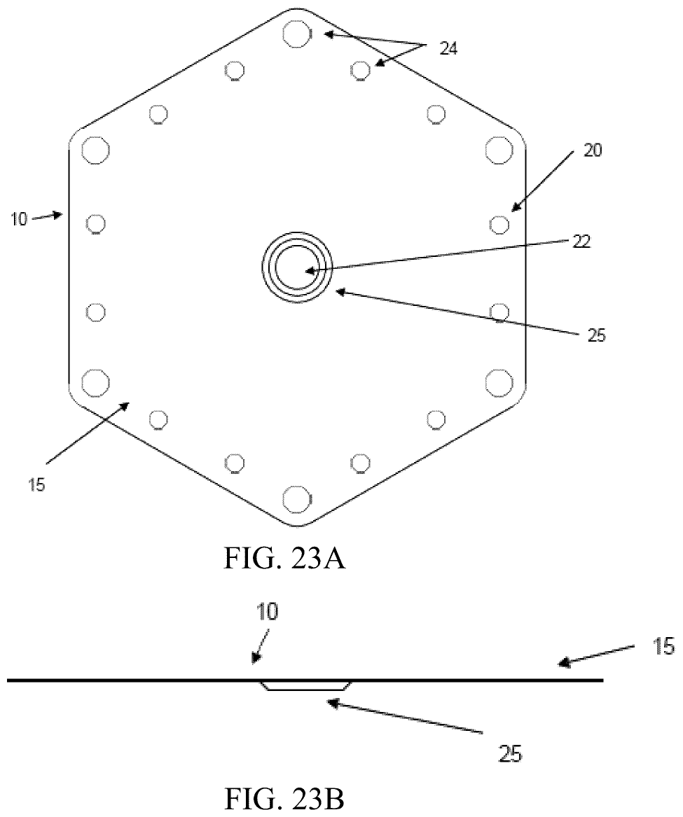

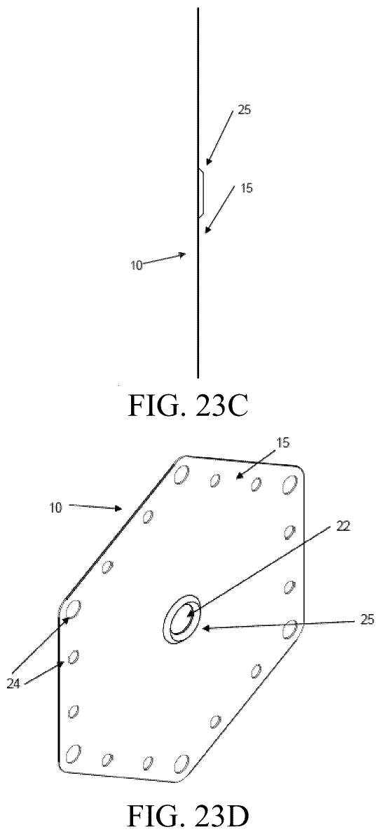

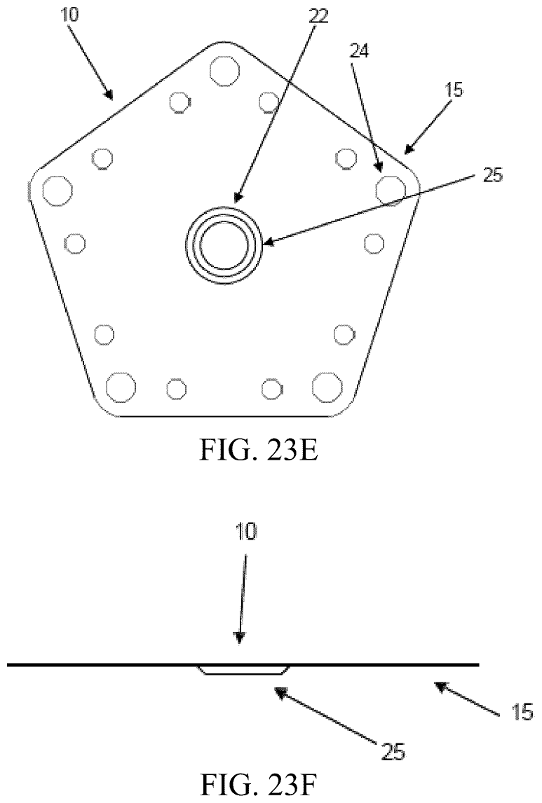

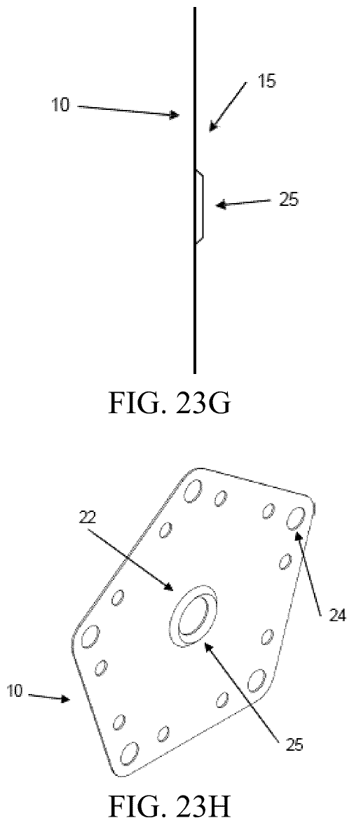

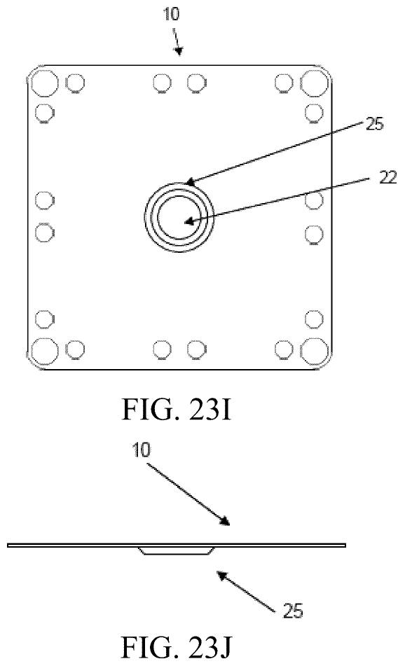

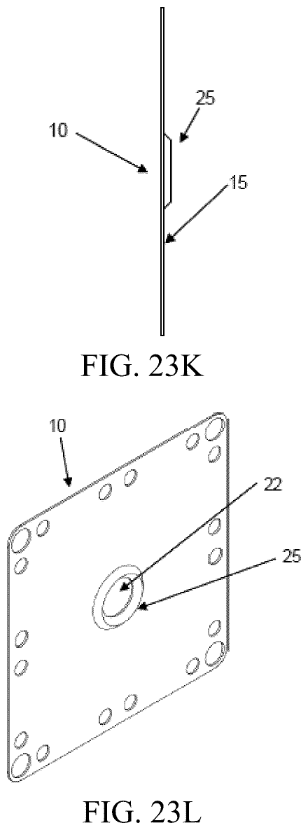

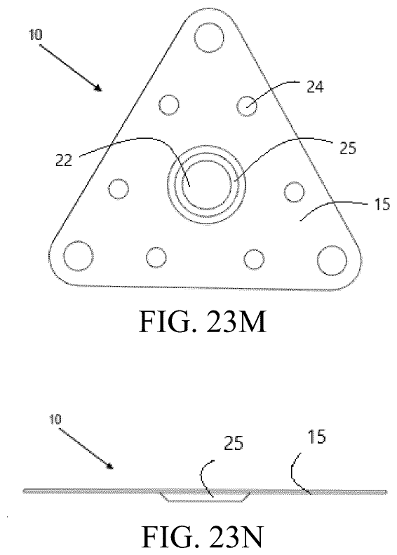

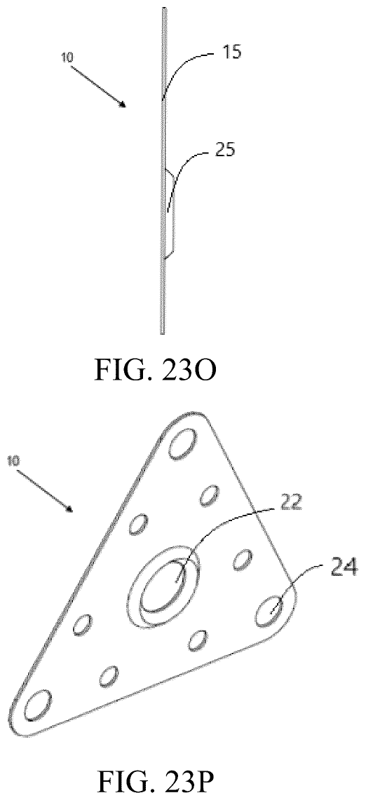

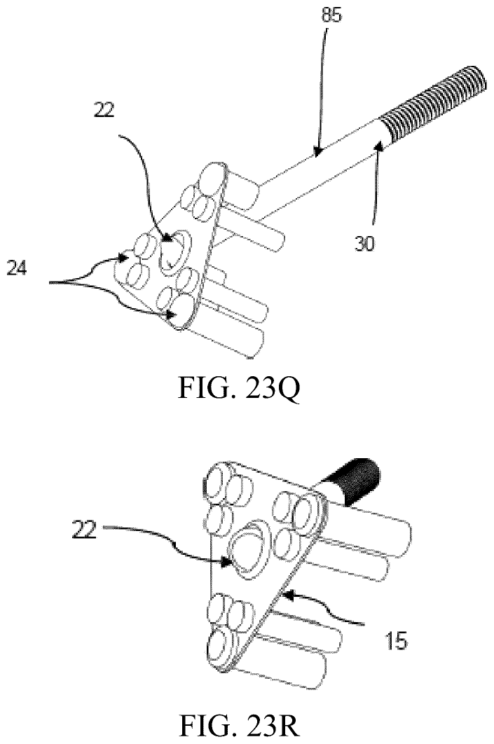

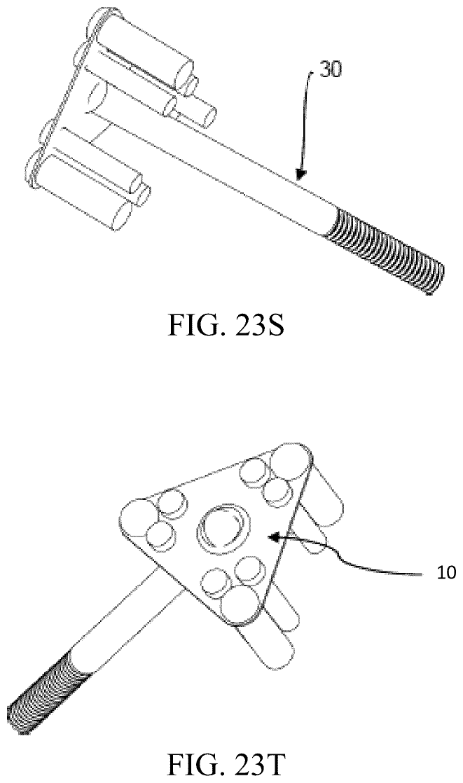

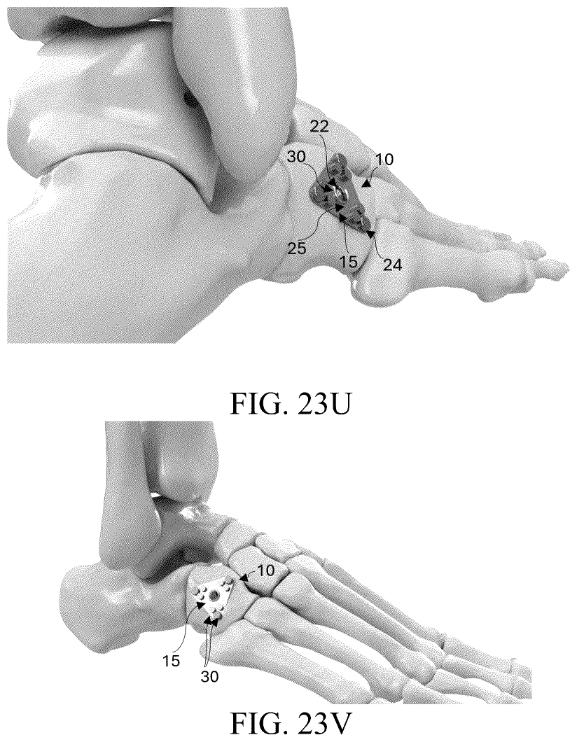

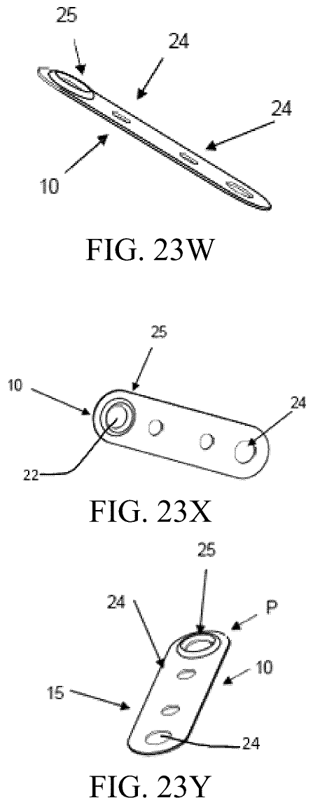

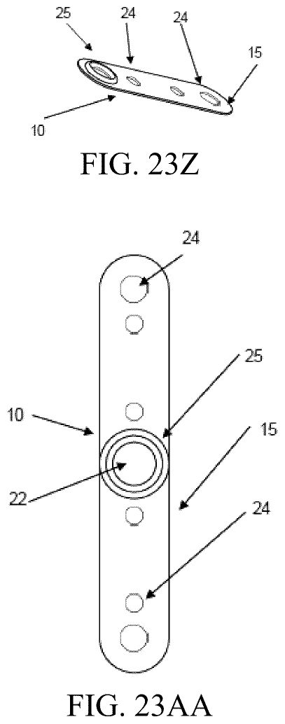

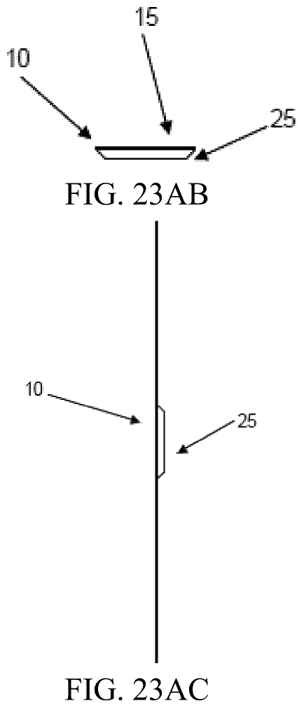

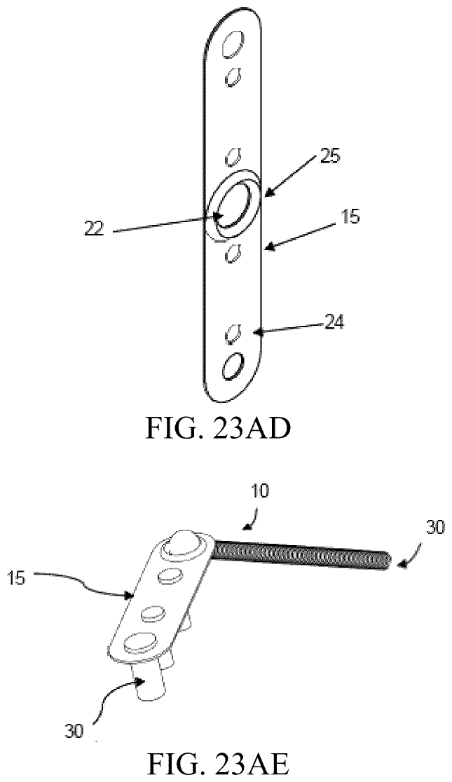

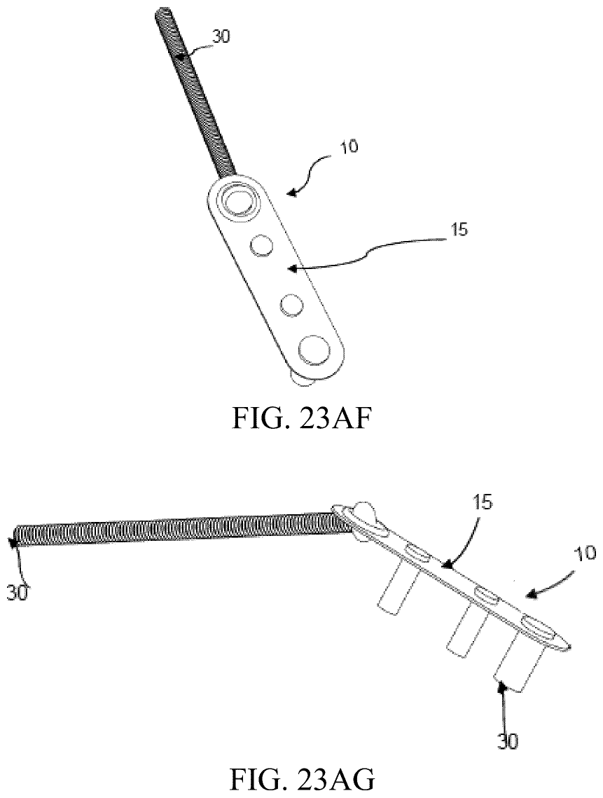

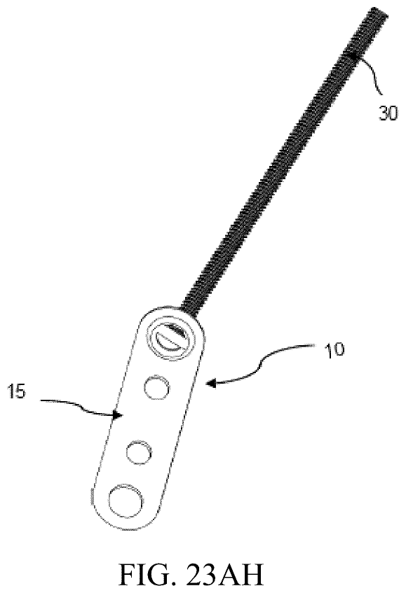

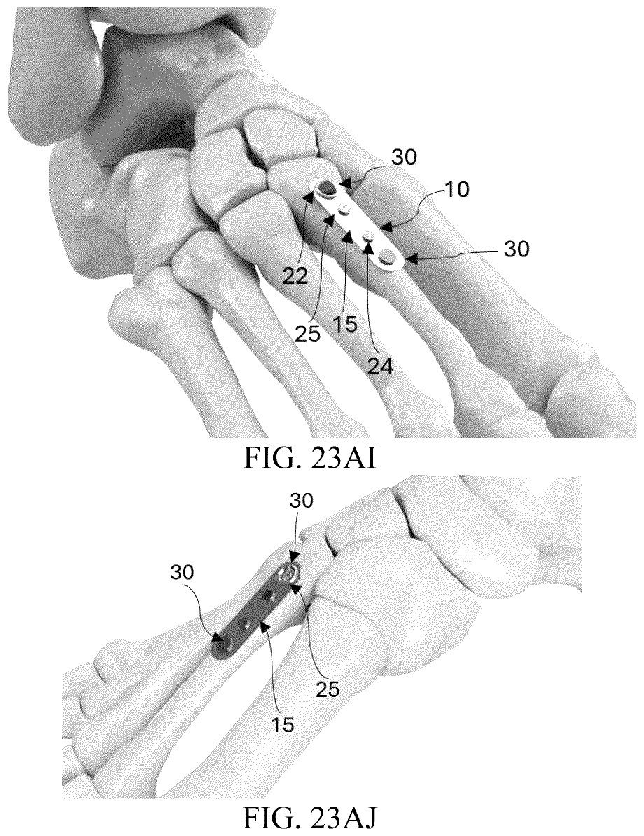

As yet an additional example of suitable shapes of the plate 15, FIGS. 23A-23AJ show some embodiments in which the plate 15 has a polygonal shape or a rounded polygonal shape. In particular, FIGS. 23A, 23E, 23I, 23M, and 23W respectively show some embodiments in which the plate 15 is hexagonal, pentagonal, square, triangular, and rectangular with rounded ends (e.g., pill shaped), thus allowing such plates to be used in a wide variety of locations (e.g., as shown in FIGS. 23U-23V and 23AI-23AJ).

In some embodiments, the salvation plate 15 (or at least a portion thereof) is flat or is mostly flat (e.g., when viewed from its proximal P or distal D ends). By way of non-limiting illustration, FIGS. 13A-13I, 14L-15D, 17A-19D, 20A-21D, and 23A-23AH show some embodiments in which the plate 15 (or a majority or at least a significant portion) of each plate is relatively flat (at least initially, before some embodiments are bent or otherwise shaped to fit one or more bones).

That said, in some embodiments, the salvation plate 15 comprises one or more raised portions, lowered portions, convex portions, concave portions, rounded portions, curved portions, or any other suitably shaped portion that allows for: increased surface area contact between one or more portions of the plate (e.g., the first face 60) and one or more bones or joints; a portion of the plate to extend into a countersunk portion of one or more bones; an edge of the plate (e.g., a proximal end P or any other suitable portion) to be flush with, to be disposed below, or to be disposed less than 1 cm (or any suitable amount less than that) above an outer surface of a bone to which the plate is attached; or that otherwise allows the plate to function as described herein. Indeed, some embodiments of the salvation plate are curved, arched, undulated, angled, or are otherwise not straight between the plates first end 40 (e.g., the proximal end P) and second end 45 (e.g., the distal end D). Moreover, in some embodiments, the salvation plate is curved, arched, undulated, angled, or otherwise not straight between the plates first lateral side 50 and second lateral side 55. By way of non-limiting illustration, FIGS. 1A-4, 10-12B, 14A-14D, 14F-14K, 19E-19H, and 22A-22B show some embodiments in which the salvation plate 15 is (or comprises a significant portion that is) rounded, curved, or angled between its first 50 and second 55 lateral sides.

Where the salvation plate 15 is rounded, curved, or angled between its first 50 and second 55 lateral sides, the salvation plate can be rounded, curved, or angled in any suitable manner that allows it to function as described herein. Indeed, in some embodiments, the salvation plate comprises a segment of a cylinder, a portion of a cone shape, any suitable radius of curvature, a shape that substantially matches an outer shape of one or more bones, a polygonal shape, or any other suitable shape. By way of non-limiting illustration, FIGS. 1A-4 show some embodiments in which the salvation plate 15 comprises an arch-shape (when viewed from the first 40 or second 45 end) or segment of a cylinder.

Although some embodiments of the plate 15 are completely straight from the first end 40 to the second end 45 of the plate (as discussed above), some embodiments of the plate comprise one or more recessed portions 27 or curved, bent, or angled portions 29. By way of non-limiting illustration, FIGS. 13B, 13F, 13G-13I, 14A, 14I, 14M, 15B-15C, 16B-16C, 17B-17C, 18A, 18C, 19B, 19D, 19F, 19H, 20B, 20C, 21C-21D, 22I-22J, and 24A-24B (among others) show some embodiments in which the plate 15 comprises a recessed portion 27 or a curved, bent, or angled portion 29. In this regard, a recessed portion of the plate can be any portion of the plate that extends past (or below): an internal or first face 60 of a body of, a majority of, a longitudinal axis of a body of the plate (or of a portion of the plate that extends from a distal end towards a proximal end of the portion of the plate that defines one or more second fastener openings), or at least a significant portion of the plate (e.g., a portion of the plate that defines one or more second fastener openings 24) so as to extend into a recess in one or more corresponding bones. Moreover, a curve, bent, or angled portion of the plate can comprise any portion of the plate that is configured to extend past (or above) an external or second face 65 of the body of, the majority of, or at least a significant portion of the plate (e.g., a portion of the plate that defines the one or more second fastener openings).

Where the plate 15 comprises one or more recessed portions 27 or curved, bent, or angled portions 29, such portions can be disposed in any suitable location with respect to the plate, including being located proximally, distally, laterally, medially, in a central position, in an off-center position, distal to one or more first fastener openings 22, proximal to one or more second fastener openings, or in any other suitable position. By way of non-limiting illustration, FIGS. 13B, 13F, 13G-13I, 14A, 14I, 14M, 15B-15C, 16B-16C, 17B-17C, 18A, 18C, 19B, 19D, 19F, 19H, 20B, 20C, 21C-21D, 22I-22J, and 24A-24B show some embodiments in which the plate 15 comprises the recessed portion 27 or the curved, bent, or angled portion 29 adjacent to a first fastener opening 22 (or anchor opening), towards the proximal end P of the plate 15. Indeed, in accordance with some embodiments, the curved, bent, or angled portion 29 is disposed at a proximal end P of the plate 15. Moreover, in some embodiments, the recessed portion 27 is disposed between one or more first fastener openings and one or more second fastener openings.

In some embodiments, the recessed portion 27 is configured to extend into a portion of a bone (e.g., a countersunk portion 70) so as to allow a fastener (e.g., a first (or anchor) fastener) to have a wide range of angular motion through a first fastener opening 22 (e.g., so as to allow the first fastener to pull the plate 15 and a bone that is coupled thereto proximally or in any other suitable direction). Where the salvation plate comprises one or more recessed portions, the recessed portions can extend any suitable amount past the first (or internal) face 60 of a distal end D (or any other suitable portion) of the plate, including to a span S (as illustrated in FIG. 22I) of between 0.1 mm and 2 cm (or within any subrange thereof). Indeed, in some embodiments, the recessed portion of the plate is configured to extend to a span S of between about 0.5 mm and 5 mm (e.g., between 2 mm and 4.5 mm) past the first (or internal) face 60 of the distal end D (or of any other suitable portion) of the plate.

Where the plate 15 comprises one or more curved, bent, or angled portions 29 that define or are adjacent to one or more first fastener openings 22 (or anchor openings), the curved or angled portions can extend any suitable distance (or height H, as shown in FIG. 22I) past the second (or external face) 65 of the plate (e.g., to allow the proximal end P of the plate 15 to allow the first fastener (e.g., an anchor fastener) to have a desired angular range of motion and to allow the proximal end of the plate to be at, below, or only slightly above adjacent bone (e.g., as shown by the bone-height BH line in FIG. 22I). Indeed, in some embodiments, the curved or angled portions extend to a height H of between 0.1 mm and 2 cm (or within any subrange thereof) past the second (or external face) 65 of the plate. For instance, some embodiments of the plate comprise curved, bent, or angled portions 29 that extend to a height H of between about 0.5 mm and 5 mm (e.g., between 2 mm and 4.5 mm) past the second (or external) face 65 of the distal end D (or any other suitable portion) of the plate.

The salvation plate 15 can be any suitable size that allows it to function as described herein. For instance, the salvation plate can be any suitable thickness, including having a thickness (T, as illustrated in FIG. 1A) between 0.001 mm and 2 cm, or within any subrange thereof. Indeed, in some embodiments, the plate has a thickness T that is between 0.5 mm and about 1 cm. In some other embodiments, the interface has a thickness T that is between 1 mm thick and 6 mm thick (e.g., to allow it to fit relatively well within a patient's body).

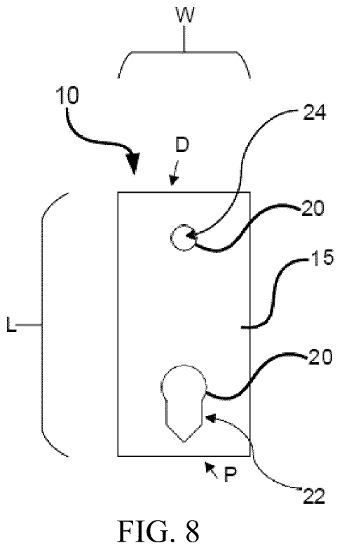

The salvation plate 15 can also have any suitable width (e.g., as shown by W in FIG. 8) that allows it to function as intended. Indeed, in some embodiments, the salvation plate has a width W that is between 5 mm and 20 cm (or within any subrange thereof). In some embodiments, for example the salvation plate has a width W that is between 5 mm and 5 cm. For instance, in some embodiments, the salvation plate has a width W that is between 1 cm and 6 cm.

The salvation plate 15 can also have any suitable length (e.g., as shown by L in FIG. 8) that allows it to function as intended. Indeed, in some embodiments, the salvation plate has a length L that is between about 5 mm and about 30 cm (or within any subrange thereof). In some embodiments, for example, the salvation plate has a length L that is between 1 cm and 15 cm. For instance, in some embodiments, the salvation plate has a length L that is between 2 cm and 10 cm.

In some embodiments, the width W and the length L of the salvation plate 15 are substantially equal to each other. In some other embodiments, however, the width W is shorter than the length L. By way of non-limiting illustration, FIGS. 5-9 show some embodiments in which the salvation plate 15 comprises an elongated plate that is significantly longer than it is wide. In contrast, FIG. 20A shows an embodiment in which the width W of the plate 15 is greater than its length L.

Turning now to the fastener openings 20, the salvation plate 15 can comprise any suitable type of fastener openings that allow the plate to function as described herein. Indeed, in some embodiments, the plate comprises one or more first fastener openings (or anchor fastener openings) openings 22, one or more second fastener openings (or civility fastener openings) 24, or any other suitable type of fastener opening. In some embodiments, however, the plate comprises a combination of first fastener and second fastener openings. While the first and second fastener openings can have any suitable characteristic, in some embodiments, the second fastener openings provide fasteners that extend therethrough with a smaller range of angular motion than do the first fastener openings (as described in more detail below).

Where the salvation plate 15 comprises one or more first 22 or second 24 fastener openings, the salvation plate 10 can include any suitable number of each type of opening. Indeed, in some embodiments, the salvation plate comprises (or defines) 1, 2, 3, 4, 5, 6, 7, 8, 9, 10, 11, 12, or more first or second fastener openings. By way of non-limiting illustration, FIGS. 5, 21G, and 20A respectively show some embodiments in which the plate 15 comprises 1, 2, and 3 first fastener openings 22 (or anchor openings). Additionally, FIGS. 5, 7, 9, and 13D, respectively show some embodiments in which the salvation plate 15 comprises 3, 2, 1, and 14 second fastener openings 24 (or civility holes).

While (as discussed above) the salvation plate 15 can comprise any suitable number of fastener openings 20 (e.g., first 22 or second 22 fastener openings), in some embodiments, the plate comprises between 0.5 and 3 (or any subrange thereof) fastener openings per centimeter of the length L of the salvation plate. Indeed, in some embodiments, the salvation plate defines between 0.5 and 1.5 (e.g., 1) fastener openings (first fastener openings, second fastener openings, or combinations thereof) for each cm in the length L of the salvation plate.

Where the salvation plate 15 comprises one or more fastener openings 20 (e.g., first 22 or second 24 fastener openings), the fastener openings can be disposed at any suitable locations in the salvation plate. Indeed, in some embodiments, the fastener openings are disposed adjacent to the first end 40 (e.g., the proximal end P), adjacent to the second end 45 (e.g., the distal end D), adjacent to the first lateral side 50, adjacent to the second lateral side 55, at or adjacent to a center of the plate, at any suitable location between the first and second ends, at a corner or edge of the plate, at any suitable location between the first lateral side and the second lateral side, or in any other suitable location. Indeed, in some embodiments, one or more first fastener openings are disposed at or adjacent to the proximal end P of the plate. In some cases, one or more first fastener openings are disposed in a center of the plate. Additionally, in some cases, one or more first fastener openings are disposed distal to another first fastener opening.

Where one or more first fastener openings 22 are disposed at or adjacent to a proximal end P of the plate 15, such openings can be disposed in any suitable location that allows the plate to function as intended. By way of non-limiting illustration, FIGS. 13A, 13B, 20A, 21A, 21G, and 23Y show some embodiments in which one or more first openings 22 are disposed at or adjacent to the proximal end P of the plate 15 (e.g., at a flat portion, at an angled, bent, or curved portion 29, in a branched portion 23 (see FIG. 21G), or at any other suitable portion of the plate). In some such embodiments, when one or more fasteners 30 are inserted into the plate (e.g., one or more civility fasteners are inserted through one or more second fastener openings 24 or any other suitable opening) prior to insertion of an anchor fastener through a first fastener opening (or anchor opening), a fastener (e.g., an anchor fastener) can be inserted into the first fastener opening and tightened to pull or lag the plate and the attached bone proximally (or in any other suitable direction).

In some other examples that show placement of the first fastener opening 22, FIGS. 23A-23V and 23AA-23AD show some embodiments in which the first fastener opening 22 is disposed at or adjacent to a center of the plate 15. In some such embodiments, a fastener (e.g., an anchor fastener) is inserted through the first fastener opening and advanced into bone to pull the plate and any bone coupled to the plate in a desired direction (e.g., proximally).

With respect to the second fastener openings 24 (or civility holes), FIGS. 5-9, 13A, 13D, 14A, 14D, and 23E (among others) show that, in some embodiments, one or more second fastener openings 24 are disposed: distal to one or more first fastener openings 22, at the distal end D of the plate 15, near the first lateral edge 50, near the second lateral edge 55, between the first 50 and second 55 lateral edges, at a corner of the plate 15, down the center of the plate, or in any other suitable location.

Where the salvation plate 15 comprises one or more fastener openings 20 (e.g., first 22 or second 24 fastener openings), the fastener openings can have any suitable characteristic that allows a fastener 30 to pass therethrough to couple the salvation plate to one or more bones or joints. For instance, the fastener openings can have any suitable shape that allows them to perform their intended function, including being circular, being cylindrical, being keyed, having multiple widths or diameters, being funnel-shaped, being cup-shaped, flaring, tapering, being square, being rectangular, being polygonal, being symmetrical, being asymmetrical, or having any other suitable shape. By way of non-limiting illustration, FIG. 5 shows an embodiment in which the first 22 and second 24 fastener openings 20 are circular.

Additionally, FIG. 8 shows an embodiment in which at least one of the openings (in this case, a first fastener opening 22 is (though any other fastener opening could be)) keyed or comprises multiple widths. While such a keyed shape can perform any suitable function, in some embodiments, the broader portion of the opening is configured to allow a fastener (or anchor) head or other component to pass therethrough. Additionally, in some such embodiments, the narrower portion of the fastener opening is configured to allow a shaft of a fastener 30 to pass therethrough, while preventing the head or any other suitable portion of the fastener from passing through the narrower portion. Thus, in some embodiments, when a fastener is coupled with a bone, the salvation plate can be placed on the fastener such that a head of the fastener passes through the broader portion of the fastener opening. Then the plate can be slid such that the shaft of the fastener is disposed in the narrower portion of the fastener opening. At that point, the fastener can be tightened down or one or more other fasteners can be inserted or tightened down in the plate to lock the salvation plate in place with respect to one or more bones.

As another example of a suitable characteristic of the fastener openings (e.g., the first 22 and second 24 fastener openings), the fastener openings can be any suitable sizes that allow them to function as intended. Indeed, in some embodiments, the openings have an inner diameter that is between 0.5 mm and 2.5 cm (or within any subrange thereof). Indeed, in some embodiments, the fastener openings have an inner diameter that is between 2 mm and 1.5 cm (e.g., between 3 mm and 5 mm).

Where the plate 15 comprises both the first 22 and the second 24 fastener openings, the various fastener openings can be suitable size with respect to each other. For instance, the first fastener openings can be larger than, smaller than, or equal in size to one or more second fastener openings. Indeed, in some embodiments, the first fastener openings are larger than the second fastener openings (e.g., to allow the first fastener opening to provide an anchor fastener with a larger range of angular motion than the second fastener opening can provide its fasteners 30 (or civility fasteners). In such embodiments, the first fastener openings can be any suitable amount larger than the second fastener openings, including being between 1 percent and 1500 percent larger in diameter (or width), or within any subrange thereof, of the diameter (or width) of a second fastener opening. Indeed, in some embodiments, the first fastener opening is between 20 percent and 500 percent larger (or within any subrange thereof) than at least one of the second fastener openings in the plate 15.

In some embodiments, the fastener openings 20 (e.g., the first 22 or second 24 fastener openings) are configured to limit a range of motion or an angle of ingress of a corresponding fastener 30 that extends therethrough. While in some embodiments, all of the fastener openings (e.g., the first and second fastener openings) are configured to limit an ingress angle of a fastener to the same extent, in some embodiments, one or more openings (e.g., one or more first fastener openings) are configured to limit an ingress angle of a fastener 30 (e.g., an anchor fastener) to a lesser extent than do one or more second fastener openings limit an ingress angle of the civility fasteners in the salvation plate 15.

By way of non-limiting illustration, FIGS. 1A, 1B, 5-9, 13A, 13G, 14A, 14L, 19A, 21A, and 23A (among others) show some embodiments in which the first fastener openings 22 are configured to allow for a fastener 30 (e.g., and anchor fastener) to have a greater range of angular motion as it passes through the first fastener opening 22 than would the fasteners (e.g., civility fasteners) that correspond one or more second fastener openings in the plate 15. For instance, FIGS. 1A and 1B show that, in some embodiments, the first fastener opening 22 allows for a significantly greater range of angular motion (or ingress angles) (as illustrated by angle a) than do one or more second fastener openings 24 (as illustrated by angles b and c). Thus, in some embodiments, the first fastener opening allows its corresponding fastener (or anchor) to be angled at a wide variety of angles to allow the fastener to capture bone that it could not, were the fastener's ingress angle more restricted.

The fastener openings 20 (e.g., the first 22 and second 24 fastener openings) can allow for their corresponding fasteners 30 to have any range (or maximum range) of angular motion as the fasteners enter or ingress into the openings and the bone covered by or adjacent to the salvation plate 15. Indeed, in some embodiments, the fastener openings are each configured to allow a tip of a fastener to pass through a fastener opening at an angle (e.g., as illustrated by angle a, b, or c in FIG. 1A) that is between 0 degrees and 230 degrees (or within any subrange thereof) in any suitable direction (e.g., proximally, distally, laterally, etc.). Indeed, in some embodiments, the first fastener openings 22 are each configured to allow a fastener (or anchor) disposed therein to move through an angular range of motion (or a maximum range of motion) that is between 1 degrees and 230 degrees, or within any subrange thereof, with respect to a portion of the first (or internal) face 60 of the plate that: is disposed distal to the first fastener opening 22 (see e.g., FIG. 13H); is disposed distal to a recessed portion 27; is disposed distal to a bent, curved, or angled portion 29 of the proximal end of the plate; defines one or more second fastener openings 24, or that would otherwise serve as a reference point for a pivot range of the fastener. In some embodiments, for instance, the first fastener opening provides its corresponding fastener (or anchor) with a range of angular motion that that allows the fastener (or anchor) to have an angle, with respect to a flat portion, a straight portion, or a longitudinal axis of at least a portion of the first (or internal) face 60 of the plate that is disposed distal to the first fastener opening 22, that is any suitable angle that is greater than 90 degrees, greater than 100 degrees, greater than 120 degrees, greater than 150, or even greater than 160 degrees. By way of non-limiting illustration, FIG. 13H shows an embodiment in which the first fastener opening 22 and plate 15 allow the fastener 30 (an anchor 85) to extend into bone (not shown in FIG. 13H at an angle of 180 degrees±10 degrees with respect to the first face 60 of the plate that is disposed distal to the first fastener opening 22. Thus, in some embodiments, the first fastener opening allows the described system to pull or lag one or more bones that are coupled to the plate in any suitable direction (i.e., proximally) in a way that would likely not be possible without some embodiments of the described system 10.

In some embodiments, the first fastener opening 22 is configured to provide its corresponding fastener 30 (anchor 85) with a maximum angular range of motion (as illustrated by angle a in FIG. 1A, with the first fastener opening 22 or a fastener head disposed therein serving as vertex) with a range of more than 10 degrees and less than 230 degrees (or within any subrange thereof) from its maximum angle in a first direction (e.g., proximally, distally, in a first lateral direction, in a second lateral direction, or in any other suitable direction), as illustrated by line α in FIG. 1A, to is maximum angle in the exact opposite direction, as illustrated by line β in FIG. 1A. Indeed, in some such embodiments, the first fastener opening allows the fastener (or anchor) to have a range of angular motion (from the vertex of the range of motion) of more than 10 degrees and less than 90 degrees (e.g., to have a maximum range of angular motion that is greater than 25 degrees, greater than 35 degrees, greater than 45 degrees, or any suitable amount greater than 25 degrees and less than 230 degrees).

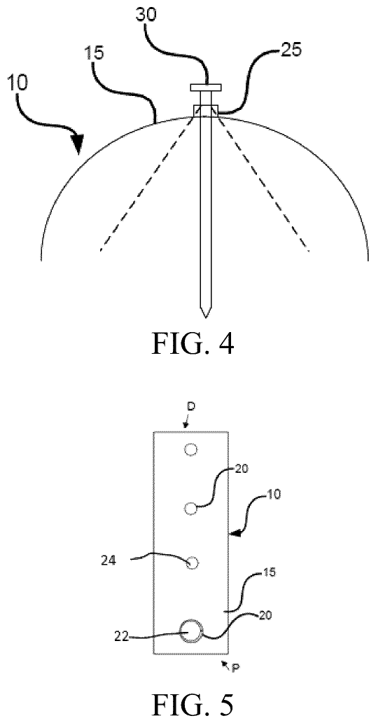

With regard to the foregoing, the various first fastener openings 22 and plate 15 can provide one or more corresponding fasteners 30 (or anchors 85) with the broad range of angular motion (as discussed above) in any suitable manner, including by: defining the first fastener opening in a curved, bent, or angled portion 29 of the plate; having the inner diameter of the first fastener opening be relatively large with respect to the outer diameter of the fastener 30 that passes through such opening; having the orifice of the first fastener opening be: cylindrical, flared from the second face 65 towards the first face 60 (e.g., as shown in FIG. 2), funneled from the second face 65 towards the first face 60 (e.g., as shown in FIG. 14D), hourglass or venturi shaped (as shown in FIG. 11), cup-shaped, or be of any other suitable shape; having a fastener spacer 25 that is configured to extend an orifice to the first fastener opening (or any other opening) away from the second face 65 (e.g., as shown in FIG. 4) to increase an amount of variation in the maximum ingress angle of the fastener; having an undersurface of a head of the fastener (or anchor) that is configured to contact a portion of the plate that defines the first fastener opening be tapered, rounded, semispherical, or to have any other suitable shape that helps to provide the fastener (or anchor) with the described angular range of motion; or by any other suitable means.

In some embodiments, the inner diameter (or width) of the first fastener opening 22 is between 10 percent and 99 percent (or within any subrange thereof) of the diameter (or width) of the head of the corresponding fastener. Indeed, in some embodiments, the inner diameter (or width) of the first fastener opening 22 is between 60 percent and 95 percent (or within any subrange thereof) of the diameter (or width) of the head of the corresponding fastener, thus giving the fastener a relatively large angular range of motion.

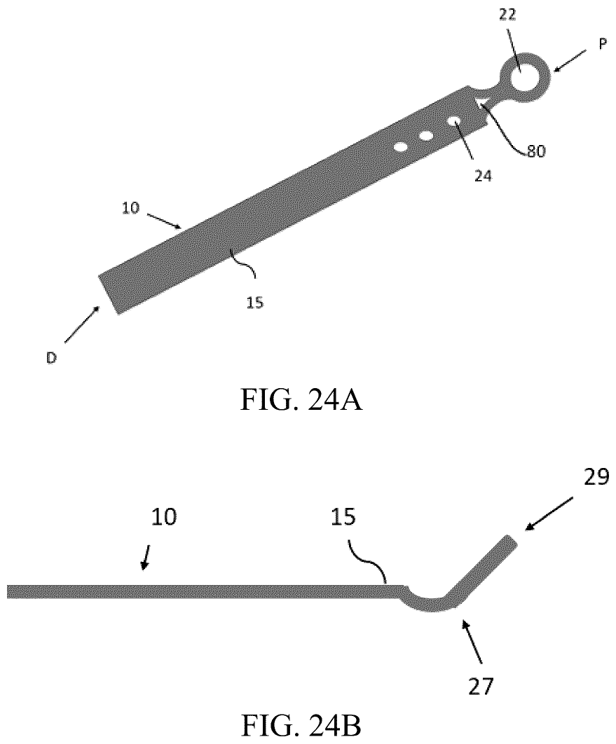

In some embodiments, to provide a fastener 30 (e.g., an anchor 85) with a relatively large range of angular motion, the plate 15 defines one or more grooves, recesses, or other channels that are configured to receive the fastener's head or to otherwise help the plate provide the fastener with a relatively large range of angular motion in the first fastener opening 22. In such embodiments, the plate can define such channels in any suitable location. By way of non-limiting illustration, FIGS. 13A, 13D, 13F, 13G, and 24A show some embodiments in which the plate 15 defines one or more channels 80 distal or adjacent to one or more first fastener openings 22.

As mentioned above, the second fastener openings 24 (or civility holes) can provide their corresponding fasteners 30 (or civility fasteners, meaning fasteners that pass through the second fastener opening) with any suitable range of angular motion, including as described above. In some embodiments, however, the second fastener openings are configured to provide their corresponding fasteners with smaller maximum angular ranges of motion than do the first fastener openings. Indeed, the second (or civility) fastener openings can provide their respective fasteners (or civility fasteners) with any smaller maximum range of motion than what the first fastener openings provide their corresponding fasteners (or anchors). Indeed, in some embodiments, the second fastener openings are configured to provide their fasteners with a maximum range of motion that is between 1 degree and 239 degrees (or within any subrange thereof) smaller than the maximum range of motion provided by the first fastener openings. Indeed, in some embodiments, the second fastener openings provide their fasteners with a range of motion that is at least 10 degrees, 20 degrees, 30 degrees, 40 degrees, or any other suitable amount that is smaller than the range of motion provided to the anchor fasteners by the first fastener openings. In this regard, while the second fastener openings and their corresponding civility fasteners can perform any suitable function, in some embodiments, their purpose is to securely couple the plate to one or more bones such that when the fastener or anchor 85 in a first fastener opening 22 is tightened to pull the plate (e.g., proximally), the fasteners in the second fastener openings (or the civility fasteners) lock the plate to the bone(s) such that the bone(s) move with the plate.

In some embodiments, the second fastener opening 24 is configured to provide its civility fasteners with a maximum angular range of motion (as illustrated by angles b or c in FIG. 1A, with the second fastener opening 24 or a fastener head disposed therein serving as vertex) with a range of between 0 degrees and 35 degrees (or within any subrange thereof) from its maximum angle in a first direction (e.g., proximally, distally, in a first lateral direction, in a second lateral direction, or in any other suitable direction), as illustrated by line δ in FIG. 1A, to is maximum angle in the exact opposite direction, as illustrated by line ϵ in FIG. 1A. Indeed, in some such embodiments, the second fastener opening allows its corresponding civility fastener to have a maximum range of angular motion (from the vertex of the range of motion) of less than 20 degrees (e.g., less than 15 degrees).

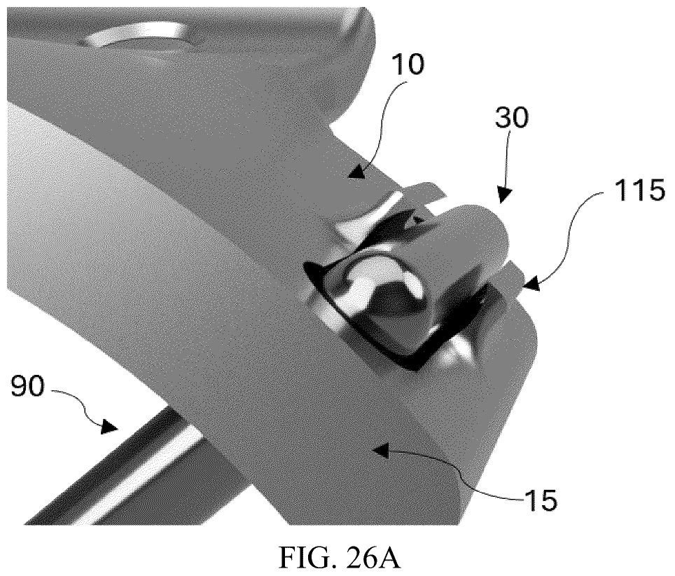

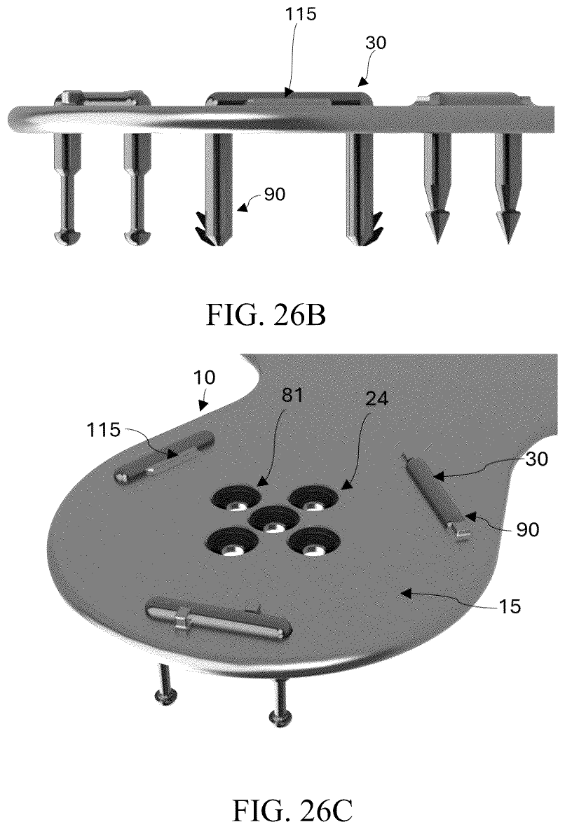

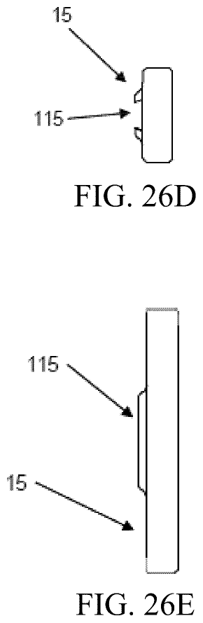

With regard to the foregoing, the various second fastener openings 24 and plate 15 can provide one or more corresponding civility fasteners with a limited (or reduced) range of angular motion (as discussed above) in any suitable manner, including by: having relatively tight tolerances between the inner diameter of the second fastener opening and the outer diameter of the corresponding civility fastener; having one or more recesses 75 defined in the plate that are configured to receive or capture a head or upper portion of the corresponding fastener (e.g., as illustrated in FIGS. 13A, 13I, 14D, and 17D); having a contoured recess in the plate that matches the contours of the fastener's head; having the plate comprise a threaded engagement 81 that couples with threads on an underside of the fastener's head (e.g., as shown in FIGS. 26C and 30); or in any other suitable manner.

In some embodiments in which the plate 15 comprises more than one second fastener openings 24, the corresponding fasteners 30 (or civility fasteners) can pass through the plate at any suitable angle or angles with respect to each other. Indeed, in some embodiments, the various civility fasteners that pass through the second fastener openings run parallel to each other, run at diverging angles with respect to each other, run at converging angles with respect to each other, or are disposed in the bone in any other suitable fashion with respect to each other. By way of non-limiting illustration, FIGS. 14B and 14C show some embodiments in which two or more of the fasteners 30 (e.g., civility fasteners that pass through second fastener openings) are configured to extend through the plate (e.g., due to the shape of the plate, the shape or size of the second fastener openings, or for any other reason) at converging angles with each other. In some such embodiments, the fasteners in the second fastener openings can ensure that the plate does not become unintentionally dislodged from one or more bones.

Where two or more second fastener openings 24 are configured to direct two or more fasteners 30 (e.g., civility fasteners) towards each other at converging angles, the fasteners can be directed towards each other at any suitable angle, including at an angle that is between 1 degree and 110 degrees (or within any subrange thereof). By way of non-limiting illustration, FIG. 14C shows an embodiment in which two or more fasteners 30 (e.g., civility fasteners) can be disposed at converging angles that are less than 90 degrees (as shown by angle z) or less than 45 degrees (as shown by angle y).

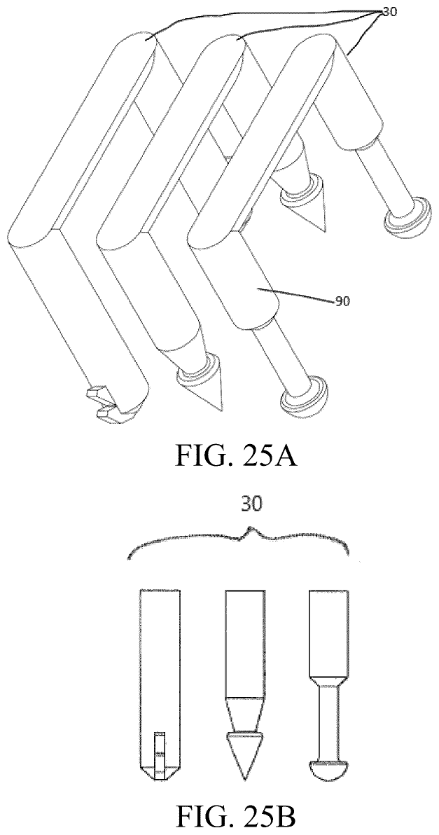

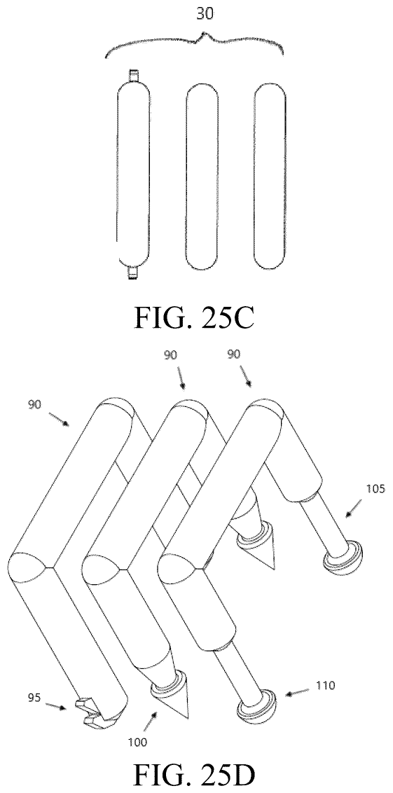

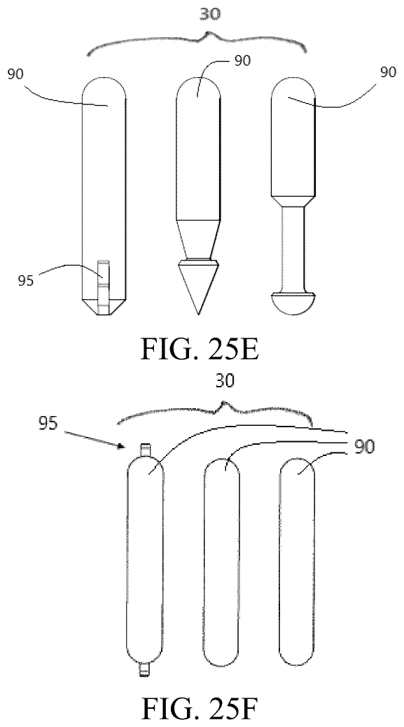

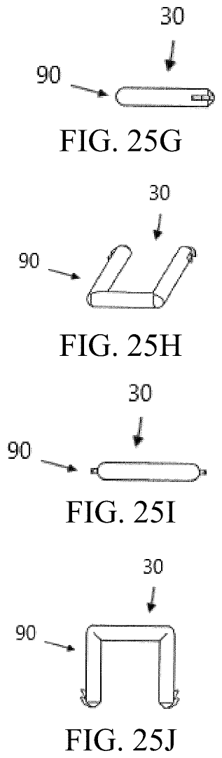

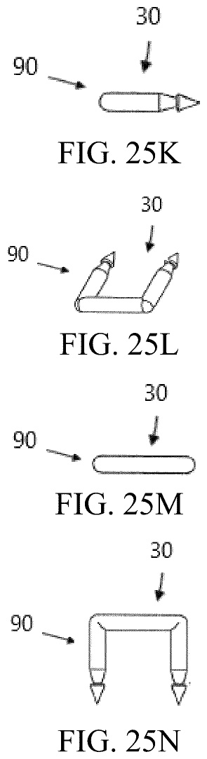

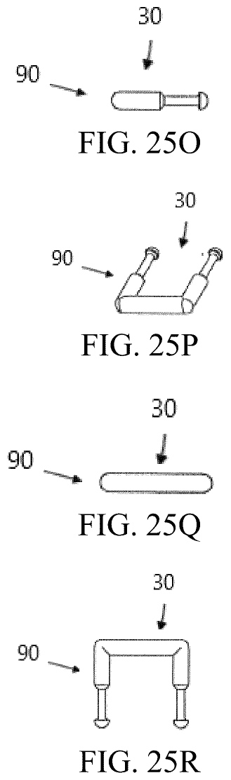

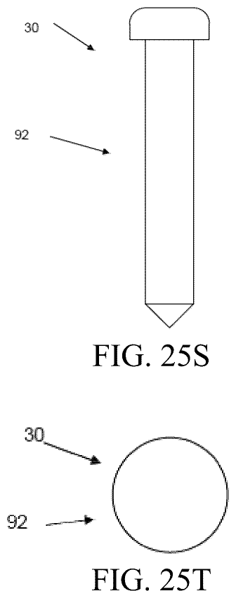

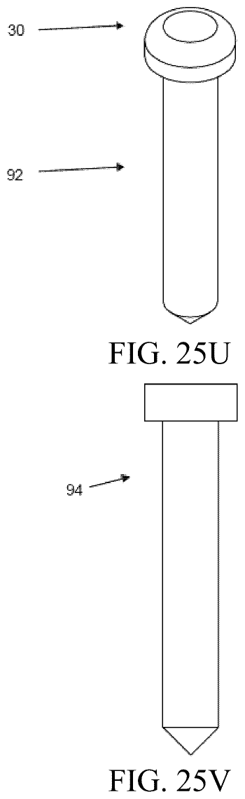

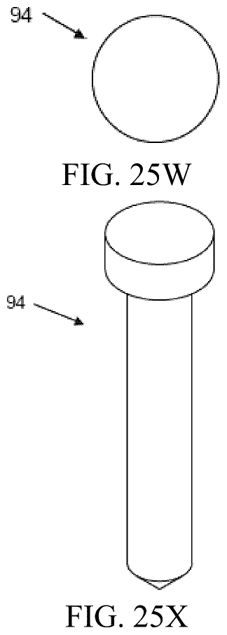

With reference now to the fasteners 30 themselves, the described salvation plate 15 can be coupled to one or more bones through the use of any suitable type of fastener. In this regard, some examples of suitable fasteners include one or more screws, bolts, lag bolts, threaded couplers, nails (e.g., round top nails, wires, or pins 92, as shown in FIGS. 25S-25T; flat top nails, wires, or pins 94, as shown in FIGS. 25V-25X; or any other suitable types of nails), pins, anchors, wires, Kirschner wires, k-wires, Steinmann pins, single trocar pins, single diamond pins, single trocar screws, single diamond screws, barbs, hooks, catches, clips, spikes, clasps, grapnels, clamps, staples, or any other suitable type of fastener that is configured to couple the salvation plate to one or more bones. By way of non-limiting illustration, FIGS. 1B and 3 show some embodiment in which the fasteners 30 (e.g., of the first 22 or second 24 fastener openings) each comprise a nail, pin, or wire. Moreover, FIG. 14A shows that, in some embodiments, the fasteners 30 (e.g., the civility fasteners that pass through the second fastener openings 24) comprise one or more screws or staples. Additionally, FIG. 13H shows an embodiment in which the fastener 30 of the first fastener opening 22 (or anchor fastener 85) comprises an elongated bolt that is configured to be directed into a viable location (or target) to grab into (or obtain “purchase”) and to be twisted to pull the plate and a bone coupled thereto towards the target.

Where the system 10 comprises one or more screws, the screws can have any suitable characteristic that allows them to function as intended (e.g., for an anchor screw passing through the first fastener opening 22 to pull the plate 15 as the screw is tightened; for a civility screw passing through the second fastener opening 24 to secure the plate to the bone; etc.). Indeed, in some embodiments, the screw (e.g., an anchor 85) has a relatively wide flat thread pitch (e.g., as shown in FIG. 13G). In some embodiments, however, the fasteners (e.g., the anchor or civility fasteners) have relatively fine threads.

Moreover, in some cases in which the system 10 comprises one or more screws, the screws have any suitable type or types of torque limiting factors (e.g., twist-off portions, perforated portions, or any other suitable torque limiter) that allows a user to know when the screw has enough purchase (e.g., when it is in tight enough) that it will not pull out. Furthermore, in some cases, the screw or other fastener is configured to not twist past a chosen foot-pound limit. Additionally, in some cases, one or more additional screws (and/or other fasteners) are used to split the load of the plate over a greater area so as to reduce the foot-pounds needed per screw.

In order to ensure that the various fasteners 30 (e.g., anchor and civility fasteners) do not back out of the plate 15 unintentionally, the plate or the fasteners themselves can have any suitable component or characteristic that is configured to keep them in the bone or plate. Indeed, in some embodiments, the fasteners comprise one or more spines, catches, spurs, collars, projections, expansions, pins, barbs, threaded engagements, adhesives, prongs, spikes, or any other suitable features that help secure the fasteners to the plate or to one or more bones. By way of non-limiting illustration, FIG. 25D shows that, in some embodiments, one or more of the fasteners (e.g., staples 90 or any other suitable fastener) comprise one or more barbs 95, sharped tips with a catch 100, narrowed portions 105, or rounded tips with a catch or spur 110.

In some embodiments, the plate 15 itself is configured to help retain one or more of the fasteners 30. In such embodiments, the plate can retain the fasteners in any suitable manner, including through the use of one or more frictional engagements, threaded engagements, non-threaded engagements, barbs, catches, spines, hooks, snaps, clasps, clips, hasps, spurs, fastener catches, or any other suitable mechanisms. By way of non-limiting illustration, FIGS. 26A-30 show some embodiments in which the plate 15 comprises one or more non-threaded engagements (e.g., catches 115) that are configured to secure a fastener 30 in place once the fastener has been properly seated in the plate and the fastener has been forced past the catch to be seated in a first 22 or second 24 fastener opening. In some other non-limiting illustrations, FIGS. 26C and 30 show some embodiments in which the plate 15 comprises a threaded engagement 81 that is configured to couple with threads on an underside of the fastener's head to further help retain the fastener in place.

In addition to the aforementioned components, the described salvation plate system 10 can comprise any other suitable component or characteristic that allows it to function as intended. In one example, the system (e.g., the plate 15, the fasteners 30, etc.) include one or more antimicrobial materials, such as one or more: types of zirconia; metallic elements (e.g., silver (Ag), copper (Cu), zinc (Zn), and any other suitable metallic elements); types of cerium, bismuth, fluorine, silicon, graphene, hydroxyapatite, or other suitable antimicrobial materials; antibiotics; antimicrobial peptides; chlorhexidine; synthetic polymers; polyethylene glycol; chitosan, polylactic acid, cellulose, hydrogels, or any other suitable materials that can carry antimicrobial drugs; or any other suitable biocompatible antimicrobial materials that comprise, are coated on, impregnated in, incorporated with, or otherwise used with one or more components of the salvation plate system 10.

In another example, while the system 10 is described herein as being used to compress or move bones in a proximal direction, any suitable portion of the system can be reoriented in any suitable manner (e.g., rotated such that the portions referred to herein as being proximal are now distal, and vice versa) to allow the system to pull or compress bone in any other suitable direction (e.g., distally).

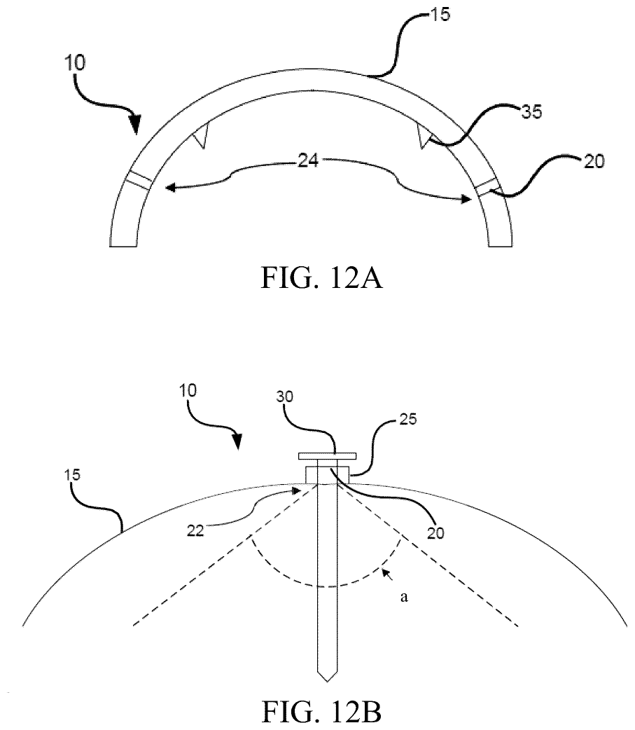

As another example, some embodiments of the salvation plate 15 comprise one or more barbs, spikes, posts, catches, protuberances, pins, posts, or other projections that extend from the first face 60 of the salvation plate. By way of non-limiting illustration, FIG. 12 shows that, in some embodiments, one or more projections 35 extend from a convex first surface 60 of the salvation plate 15 to help secure the salvation plate to one or more bones.

In addition to the aforementioned features, the described system 10 can include any other suitable feature. Indeed, in some embodiments, the second surface 65 of the salvation plate 15 includes one or more recesses 75 to allow a portion of a staple 90 or other fastener 30 to be seated in the second surface to help reduce rubbing between a portion of the fastener and tissue that contacts the second surface.

Additionally, in accordance with some embodiments, the use of fastener 30 (e.g., an anchor) through the first fastener opening 22, which provides the fastener with a large degree of angular movement, can allow the plate 15 to be properly anchored in a location in which some competing devices could not.

As another example of a suitable feature, some embodiments of the plate 15 are relatively rigid and not configured to be bent for use. In some other cases, however, one or more portions of the salvation plate comprise one or more materials or portions that are somewhat malleable. In some such embodiments, when the plate is put into place in a patient, the plate is bent (e.g., by hand, with a tool, or in any other suitable manner) to help the plate better match the contours of one or more bones.

As another example, while the salvation plate system 10 is shown herein to be used with bones in the feet, the described system can be used with any other suitable bones and in any other suitable portion of a patient's body. Indeed, in some embodiments, the system is coupled to one or more parts of one or more tarsals, metatarsals, phalanges, carpals, metacarpals, humeri, femurs, scapulae, pelvises, tibias, fibulas, radii, ulnae, or any other suitable bone or bones.

In yet another example, although some embodiments of the plate 15 comprise one or more first fastener openings 22 that are disposed at or adjacent to a proximal end P of the plate, in some other embodiments, the plate defines one or more first fastener openings at or adjacent to its distal end D. Thus, in some embodiments, instead of being used to pull one or more bones proximally, the plate is configured (and is used) to pull one or more bones (e.g., a distal portion of a fibula) distally (e.g., as mentioned above).

The described salvation plate system 10 and its various components can be made in any suitable manner. In this regard, some non-limiting examples of methods for making the described salvation plate 15 and fasteners 30 include molding, stamping, punching, drilling, bending, straitening, cold heading, heat treating, thread rolling, tapping, dying, cutting, fitting, welding, melting, forming through additive manufacture, CNC milling, and otherwise forming and assembling the various components to form the described system.

The salvation plate system 10 and its various components can comprise any suitable biocompatible material or materials that allow the components to be disposed in a patient's body and that allows such components to function as intended. In this regard, some non-limiting examples of suitable materials for the salvation plate 15 and the fasteners 30 include one or more types of: stainless steel; titanium; cobalt chrome; titanium cobalt; titanium alloy; aluminum alloy; biosynthetic material; bioresorbable material; nitinol; nickel titanium alloy; tantalum; polyetherketone; cobalt chromium-based alloy; an alloy comprising vanadium, aluminum, and titanium; zirconium; zirconia; oxinium (oxidized zirconium); ceramic material; plastic material; polymer material; or any other suitable material. Indeed, in some embodiments the salvation plate and the fasteners comprise one or more metals or ceramics. More specifically, some embodiments of the plate and fasteners comprise one or more titanium alloys.

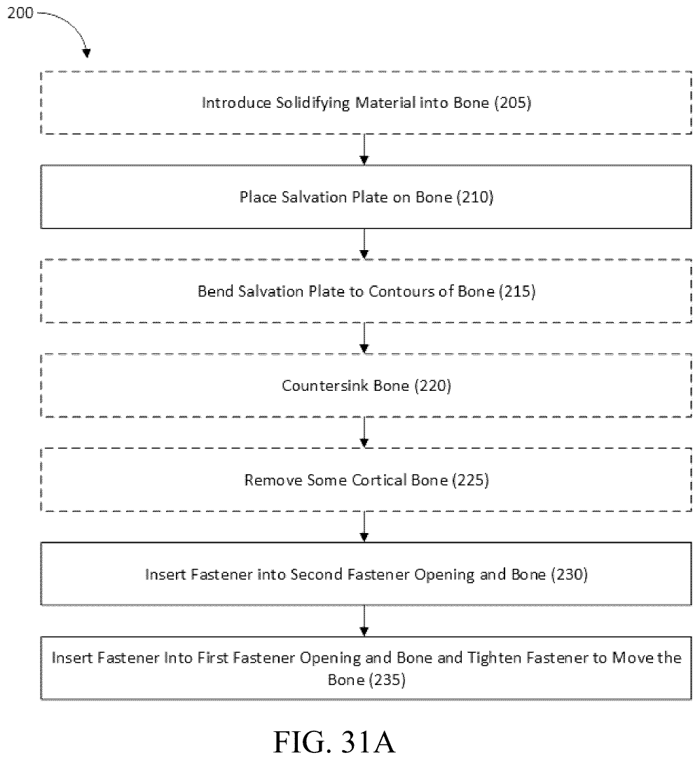

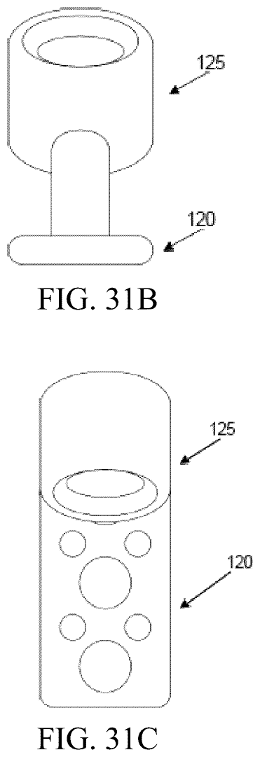

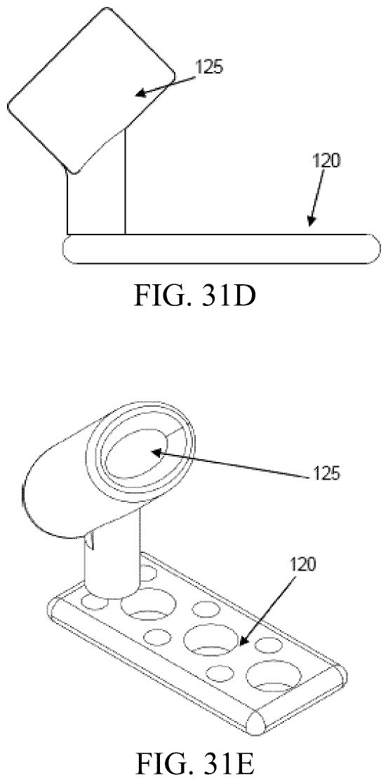

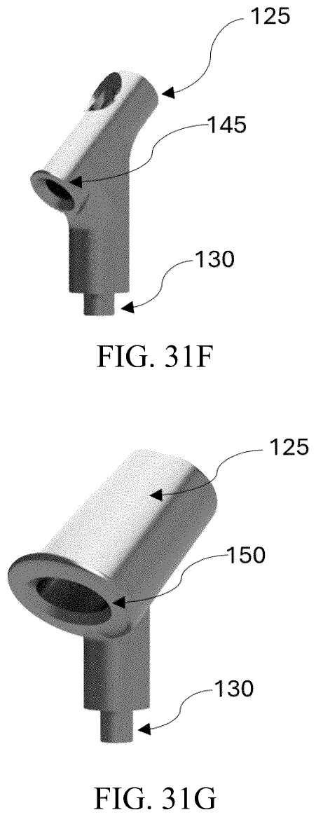

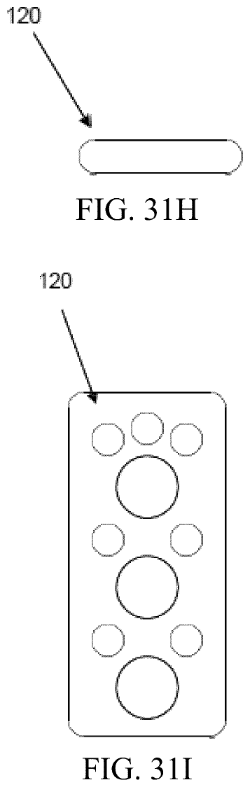

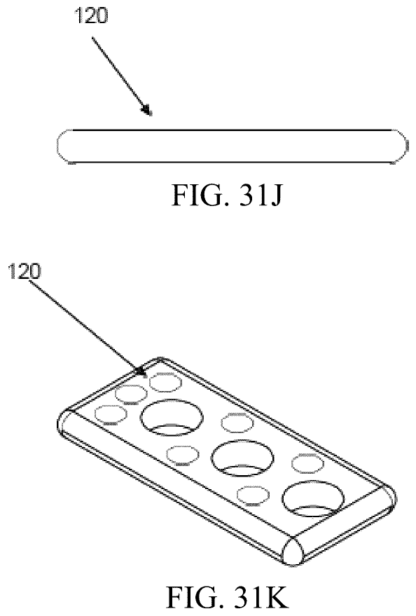

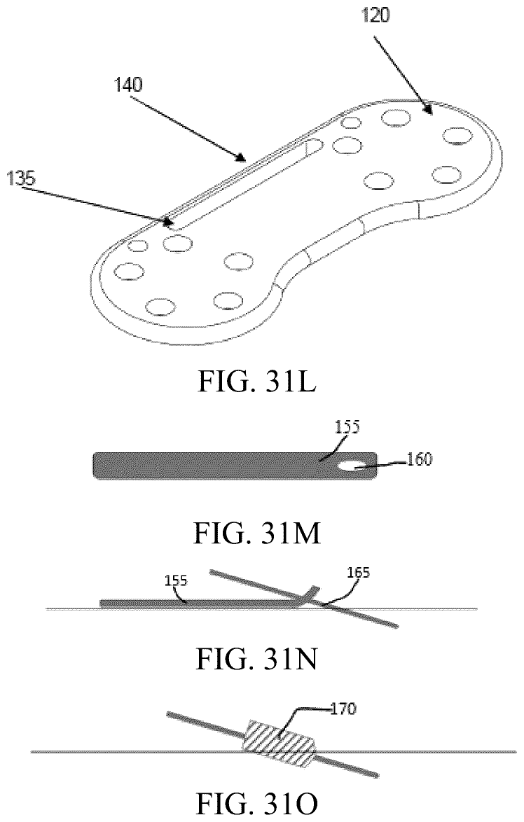

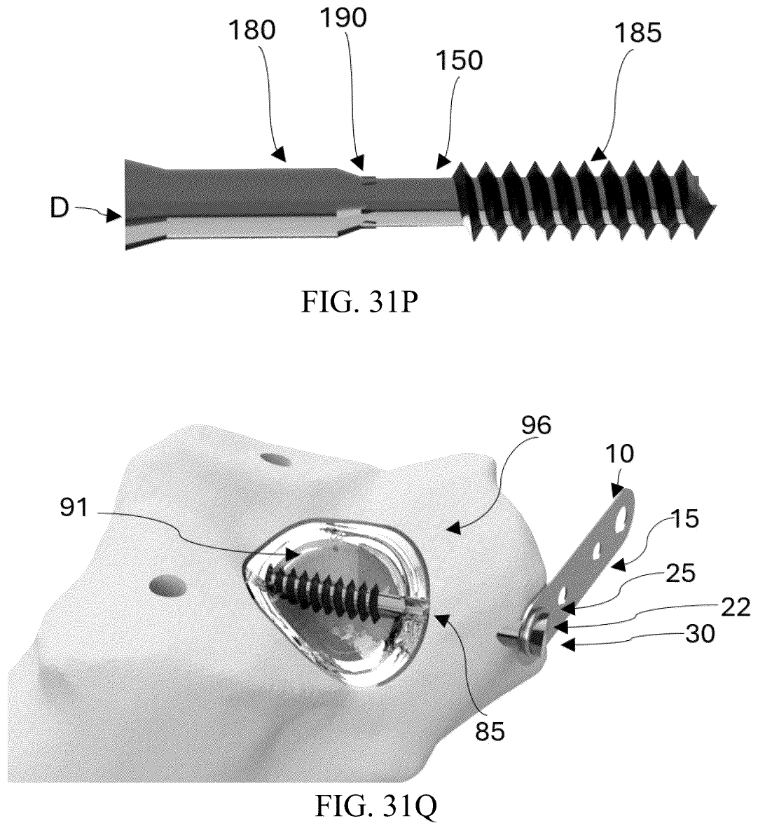

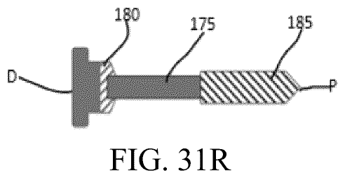

The described salvation plate system 10 can also be used in any suitable manner. By way of non-limiting example, FIG. 31A shows a flowchart depicting a method 200 for using the system. In this regard, it should be noted that any and all portions of the described method can be: rearranged and performed in any suitable order, performed sequentially, performed simultaneous, omitted, replaced, augmented, or otherwise modified in any suitable manner. In any case, FIG. 31A shows that, in some embodiments, once one or more suitable bones in any suitable portion of the body (such as in the foot, hand, or shoulder) are exposed (e.g., via surgery), the method 200 optionally begins at box 205, where one or more solidifying materials are introduced into one or more bones.

Where a solidifying material is introduced into a bone, the solidifying material can be introduced into the bone in any suitable manner, including by being injected into, packed into, or otherwise introduced into one or more bones. Moreover, where a solidifying material is introduced into a bone, the solidifying material can perform any suitable function, including providing a strong structure to receive and retain a fastener (e.g., an anchor fastener that is inserted into the bone through a first fastener opening 22 or any other suitable fastener 30); filing intramedullary or fragmentation gaps; strengthening soft, osteopenic, osteoporic, or fragmenting bone; Charcot/neurogenic-arthritic bone; and otherwise preparing the bone to receive the plate 15. By way of non-limiting illustration, FIG. 31Q shows an embodiment in which a solidifying material 91 is introduced into a bone 96, and in which a fastener 30 (e.g., an anchor 85) is driven into and secured in the solidifying material 91.

Where one or more solidifying materials is introduced into a bone, any suitable solidifying agent can be used. In this regard, some examples of such solidifying agents include one or more flowable bone sulfate/phosphate cements, acrylic bone cements, calcium phosphate bone cements, magnesium phosphate bone cements, types of polymethyl methacrylate, or any other suitable solidifying material that is biocompatible.

Continuing on to box 210, FIG. 31A shows that, in some embodiments, the method 200 continues as the plate 15 is placed over one or more bones (e.g., pieces of broken bone, collapsing bone, one or more joints, or any other suitable bone). In this regard, as there are many potential embodiments of the plate that could be used, in some cases, a medical practitioner picks the plate that best fits the then current needs.

In accordance with some embodiments, box 215 shows that the method 200 further continues as the plate 15 is optionally bent to fit one or more contours of one of more bones. While this bending can be performed in any suitable manner, in some cases, the plate is bent by hand, with a pair of pliers, with a hammer, with a tool, or in any other suitable manner.

Continuing with the method 200, box 220 shows that, in some cases (e.g., where the plate 15 comprises one or more recessed portions 27 or bent, curved, or angled portions 29), the method optionally comprises removing or otherwise countersinking an amount of bone to allow the plate to be properly seated in the patient. In such cases, the bone can be removed in any suitable manner, including through the use of one or more chisels, drill bits, grinders, burrs, guides, or in any other suitable manner.

Indeed, in accordance with some embodiments, bone is removed or otherwise countersunk through the use of one or more guides or jigs. While such a guide and jig can have any suitable configuration, FIGS. 31B-31L show that, in some embodiments, a guide 120 can be similar in shape to the plate 15 being used, while missing the recessed portion 27 or curved, bent, or angled portion 29. Additionally, FIGS. 31B-31L show that, in some embodiments, a jig 125 that is configured to receive a countersinking tool (e.g., a drill bit, burr, oscillating tool, chisel, or any other suitable tool) is coupled to the guide (e.g., via a protrusion 130 on the jig that is configured to fit within a hole 135 in the guide; via one or more fictional engagements, magnets, mechanical engagements, welds, adhesives, catches, or couplers; or in any other suitable manner). For instance, FIG. 31L shows an embodiment in which the guide 120 comprises an elongated slit 140 that is configured to receive a protrusion 130 from a jig 125 (not shown in FIG. 31L) to allow the jig to be slid back and forth in the slit to allow a tool in the jig to remove bone across the length of the slit. Additionally, while FIG. 31F shows an embodiment in which the jig 125 comprises a cylindrical orifice 145 to receive a tool and keep the tool in a single rotational position, FIG. 31G shows an embodiment in which the jig 125 comprises an elliptical shaped orifice 150 (or any other suitably shaped orifice) that allows a tool to be pivoted or otherwise moved back and forth (or in any other desired manner, based on a shape of the orifice) to countersink the desired amount of bone.

In another example of a method for removing or otherwise countersinking bone, FIG. 31M shows that, in some embodiments, a pin guide 155 that defines one or more holes 160 can be placed over a desired portion of bone to guide placement of a pin, wire, or any other suitable guide wire (e.g., a Steinmann pin 165, as shown in FIG. 31N) into the bone. In some such embodiments, a reamer, a cannulated drill bit, or any other suitable countersinking object 170 is placed over the guide wire (e.g., when the pin guide has been removed) and actuated to countersink a desired amount of bone (e.g., as shown in FIG. 31O).

Returning to the method 200 of FIG. 31A, box 225 shows that some embodiments of the method 200 optionally include removing bone (e.g., cortical bone) at or near an insertion site of the anchor fastener (or the fastener 30 that extends through a first fastener opening 22). In such embodiments, the bone can be removed in any suitable manner, including as shown in FIGS. 31M-31O. In some other embodiments, however, FIGS. 31P and 31R show that the anchor fastener 175 itself comprises a reamer, burr, countersink tool, blade, teeth, or any other suitable bone removal tool 180 (e.g., distal to threads 185 on the anchor fastener 175, proximal to a head of the bone removal tool, coupled to the head of the fastener, or at any other suitable portion of the fastener) that is configured to fit through the first fastener opening 22 such that when the anchor fastener is driven into the bone (e.g., through the first fastener opening 22, at step 235, as discussed below), the bone removal tool extends through the opening to allow the bone removal tool to remove cortical bone and to allow the anchor fastener to pull the plate tight against the bone without splitting the bone. Indeed, in some embodiments, as such a fastener is used in the first fastener opening (or any other suitable opening, such as the second fastener opening 24), the bone removing tool interfaces with the bone such that as the tool is twisted, it removes the bone as necessary to move past any cortical bone that was not removed (if any was) in countersinking to allow the plate to be pulled right against the bone (e.g., to prevent a gap between the plate and the bone or to prevent the plate from being prominent and unduly rubbing against a patient's skin or other tissue).

Where the fastener 175 (e.g., a modified anchor fastener, as shown in FIGS. 31P and R) comprises a screw/fastener having threads 185 and a bone removal tool 180, the fastener can have any suitable characteristic that allows it to secure the plate 15 to one or more bones. Indeed, in some embodiments, the bone removal tool is disposed just below (or proximal) to a head of the fastener. In some such embodiments, the head of the fastener comprises a wider diameter (or width) or is otherwise larger or shaped differently than the bone removal tool such that when the fastener is inserted into a fastener opening (e.g., a first fastener opening 22), the head prevents the fastener from being pulled through the fastener opening and instead acts to pull plate 15 to force the plate against a bone and to cause the plate to compress the bone. In some cases, the bone removal tool is funnel shaped from the fastener's head towards the fastener's shaft, is rounded, is semi-spherical, is cylindrical with a tapered end, or has any other suitable shape. By way of non-limiting illustration, FIG. 31P shows an embodiment in which the bone removal tool 180 has a cylindrical portion with a tapered end, which, in some cases, comprises teeth, blades, an abrasive surface, or any other object that is suitable for removing bone. Indeed, FIG. 31P shows an embodiment in which the fastener 175 comprises teeth 190 at an interface between the tapered end of the cylindrical portion of the bone removal tool 180 and a shaft of the fastener that extends from the tapered end of the bone removal tool 180. Additionally, FIG. 31R shows an embodiment in which a proximal side of the bone removal tool 180 tapers down to the shaft of the fastener.

Where the fastener 175 has a bone removal tool 180 and threads 185, the threads (as is the case with every other fastener 30 described herein (wherever possible) or that can be used with the system 10) can have any suitable characteristic, including having any suitable: pitch, type of lead, number of starts (e.g., 1, 2, 3, 4, 5, etc.), angle, thread-form, coarseness, fineness, consistency in pitch, variation in pitch, consistency in threading, variation in threading, or have any other suitable shape or characteristic. By way of non-limiting illustration, FIG. 31P shows an embodiment in which the fastener 175 has cancellus pitch threads that allow for aggressive interface with cancellus bone, cortical bone, solidifying material 91, or any other suitable object in the body that can provide “purchase” to the fastener.

Where the fastener 175 comprises a bone removal tool 180 (e.g., as shown in FIGS. 31P and 31R, the fastener (like all other fasteners described herein) can have any suitable diameter, including a diameter or size between 0.5 and 14 (or within any subrange thereof). Indeed, in some embodiments, the fastener has a diameter of anywhere from 5 to 7.5, inclusive.

Continuing with FIG. 31A, box 230 shows that, in some embodiments, the plate 15 is coupled to one or more bones by driving one or more fasteners 30 (e.g., civility fasteners) through one or more second fastener openings 24 and into the bone.

Once the plate 15 is properly coupled to one or more desired bones, box 235 shows that, in some embodiments, the anchor fastener (or the fastener 30 that extends through the first fastener opening 22) is driven through the first fastener opening in a desired direction (e.g., proximally or in any other suitable direction) and into one or more bones or joints to pull the plate (and any bones attached thereto) in the desired direction. Indeed, in some embodiments, by so doing, the plate and desired bone or bones can be pulled between 0.1 mm and 3 cm (or within any subrange thereof) in the desired direction.

Once the plate 15 is secured in place, it can be left in place for the duration of the patient's life.

Thus, as discussed herein, the described systems and methods relate to the treatment of bones and joints. In particular, the described systems and methods relate to a salvation plate that can be placed over one or more deteriorating, broken, diseased, malformed, collapsing, or otherwise damaged bones or joints and that can be tightened to the bones or joints with one or more screws, pins, staples, nails, bolts, threaded engagements, anchors, wires, or other fasteners. In this manner, the plate can provide compression to various pieces of the bones and joints to help them heal, fuse, mend, strengthen, and for any other suitable purpose. Additionally, in some cases, one or more screws or other fasteners that extend through the plate are configured to extend through the plate in a wide range of angles. Thus, in some cases, the salvation plate allows a screw or other fastener to be driven through the plate at an angle that allows the fastener to grab pieces of bone that could otherwise not be grabbed were the fastener not able to be inserted at that specific angle.

As the systems and methods disclosed herein are compatible with one another, the systems discussed herein can be used in practicing the methods disclosed herein, and vice versa. Accordingly, the method may further include implementing, exercising, or otherwise using any of the components discussed herein for any of their stated or intended purposes, as reasonably predictable and understood by a person of ordinary skill in the art. The systems disclosed herein can be made in any suitable manner, and they may be used in any way consistent with their operational capabilities. Moreover, in some cases, any particular element or elements of any apparatus—or portion or portions of any method—disclosed herein can be omitted.

As used herein, the singular forms “a”, “an”, “the” and other singular references include plural referents, and plural references include the singular, unless the context clearly dictates otherwise. For example, reference to a panel includes reference to one or more panels, and reference to spacer wires includes reference to one or more spacer wires. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, unless the context dictates otherwise, the term “and” may be interpreted to be “and/or”. Furthermore, the terms “including”, “having”, “such as”, “for example”, “e.g.”, and any similar terms are not intended to limit the disclosure and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An implant comprising:
a salvation plate that is configured to extend over one or more bones, wherein the salvation plate has a first end and a second end and defines:
an anchor fastener opening that is configured to allow a first fastener to extend through the anchor fastener opening such that the first fastener enters bone covered by the salvation plate at a first angle that falls within a first maximum range of angular motion; and
a first civility fastener opening that is configured to allow a second fastener to extend through the first civility fastener opening such that the second fastener enters the bone covered by the salvation plate at a second angle that falls within a second maximum range of angular motion, wherein the second maximum range of angular motion is smaller than the first maximum range of angular motion.

2. The implant of claim 1, wherein the second maximum range of angular motion is at least twenty degrees smaller than the first maximum range of angular motion.

3. The implant of claim 1, wherein the anchor fastener opening is disposed adjacent to the first end of the salvation plate, and wherein the first end of the salvation plate comprises a proximal end of the salvation plate.

4. The implant of claim 1, wherein the anchor fastener opening is disposed at least one of: (i) at and (ii) adjacent to a center of the salvation plate.

5. The implant of claim 1, wherein the salvation plate further comprises an exterior surface that comprises a raised spacer that is disposed at a perimeter of the anchor fastener opening.

6. The implant of claim 1, wherein the salvation plate further defines a second civility fastener opening that is configured to allow a third fastener to extend through the second civility fastener opening such that the third fastener enters the bone covered by the salvation plate at a third angle that falls within the second maximum range of angular motion, and wherein the first civility fastener opening and the second civility fastener opening are configured to respectively direct the second fastener and the third faster at intersecting angles towards each other.

7. The implant of claim 1, wherein the anchor fastener opening is disposed adjacent to the first end of the salvation plate, and wherein a portion of the salvation plate that defines the anchor fastener opening is at least of: (i) curved, (ii) bent, and (ii) angled past an external surface of a portion of the salvation plate that defines the first civility fastener opening.

8. The implant of claim 1, wherein the salvation plate comprises a recessed portion that is disposed adjacent to the anchor fastener opening and proximal to the first civility fastener opening, and that is configured to extend into a corresponding recess formed in the bone covered by the salvation plate.

9. The implant of claim 1, wherein the salvation plate comprises a non-threaded retainer that is configured to capture the second fastener once the second fastener is fully inserted into the first civility fastener opening.

10. The implant of claim 1, wherein the salvation plate comprises a concave internal surface and a convex external surface that is disposed substantially opposite to the concave internal surface, and wherein at least a portion of the anchor fastener opening flares as it extends from the concave internal surface towards the convex external surface.

11. The implant of claim 1, wherein the anchor fastener opening is defined at a proximal end of the salvation plate, and wherein the salvation plate further defines an elongated channel that is disposed adjacent to the anchor fastener opening and that is configured to receive a portion of a head of the first fastener.

12. An implant comprising:
a salvation plate that is configured to extend over an outer surface of one or more bones, wherein the salvation plate has a proximal end and a distal end and defines:
an anchor fastener opening that is disposed at the proximal end of the salvation plate and that is configured to allow a first fastener to extend through the anchor fastener opening such that the first fastener enters a bone covered by the salvation plate at a first angle that falls within a first maximum range of angular motion;
a first civility fastener opening that is configured to allow a second fastener to extend through the first civility fastener opening such that the second fastener enters the bone covered by the salvation plate at a second angle that falls within a second maximum range of angular motion, wherein the second maximum range of angular motion is smaller than the first maximum range of angular motion, and wherein the second maximum range of angular motion is at least 20 degrees smaller than the first maximum range of angular motion; and
a second civility fastener opening that is configured to allow a third fastener to extend through the second civility fastener opening such that the third fastener enters the bone covered by the salvation plate at a third angle that falls within the second maximum range of angular motion,
wherein a portion of the salvation plate that defines the anchor fastener opening is at least of: (i) curved, (ii) bent, and (iii) angled out past a plane of an external surface of a portion of the salvation plate that defines the first civility fastener opening, and
wherein the first civility fastener opening and the second civility fastener opening are configured to respectively direct the second fastener and the third faster at intersecting angles towards each other.

13. The implant of claim 12, wherein the salvation plate comprises at least one of: (i) a branched configuration and (ii) a lobed configuration.

14. The implant of claim 12, wherein the first maximum range of angular motion provides the first fastener with an insertion angle with more than 35 degrees of angular motion.

15. The implant of claim 12, wherein the salvation plate comprises a concave internal surface and a convex external surface that is disposed substantially opposite to the concave internal surface.

16. A method for implanting an implant, the method comprising:
obtaining a salvation plate that is configured to extend over one or more bones, wherein the salvation plate has a first end and a second end and defines:
an anchor fastener opening that is configured to allow a first fastener to extend through the anchor fastener opening such that the first fastener enters bone covered by the salvation plate at a first angle that falls within a first maximum range of angular motion; and
a first civility fastener opening that is configured to allow a second fastener to extend through the first civility fastener opening such that the second fastener enters the bone covered by the salvation plate at a second angle that falls within a second maximum range of angular motion, wherein the second maximum range of angular motion is smaller than the first maximum range of angular motion;
seating the salvation plate;
inserting the second fastener into the bone covered by the salvation plate through the first civility fastener opening; and
inserting the first fastener into the bone covered by the salvation plate through the anchor fastener opening.

17. The method of claim 16, further comprising countersinking a portion of bone that is to be disposed adjacent to the anchor fastener opening when the first fastener is inserted into the anchor fastener opening.

18. The method of claim 16, further comprising laying a guide over the bone, with the guide having a jig disposed thereon, and using the jig to countersink the bone prior to laying the salvation plate over the bone.

19. The method of claim 16, wherein a second civility fastener opening that is configured to allow a third fastener to extend through the second civility fastener opening such that the third fastener enters the bone covered by the salvation plate at a third angle that falls within the second maximum range of angular motion, wherein the first civility fastener opening and the second civility fastener opening are configured to respectively direct the second fastener and the third fastener at intersecting angles towards each other, and wherein the method further comprises driving the third fastener into the second civility fastener opening and into the bone at an intersecting angle with respect to the second fastener.

20. The method of claim 19, wherein the second fastener comprises a screw and the third fastener comprises at least one of: (i) a staple, (ii) a pin, and (iii) a wire.

21. An implant comprising:
a salvation plate that is configured to extend over an outer surface of bone, wherein the salvation plate has a proximal end, a distal end, an external surface, and an internal surface, which is configured to contact the outer surface of the bone, wherein the salvation plate defines:
an anchor fastener opening that is disposed at the proximal end of the salvation plate and that is configured to allow a first fastener to extend through the anchor fastener opening such that the first fastener enters the bone at a first angle that falls within a first maximum range of angular motion that is within 120 degrees and 230 degrees with respect to a plane the internal surface of the salvation plate that is disposed distal to the anchor fastener opening.
